(19) United States
(12) Patent Application Publication
(10) Pub. No.: US 2014/0220134 A1
(43) Pub. Date: Aug. 7, 2014

(54) METHOD FOR TREATING DIABETES WITH EXTENDED RELEASE FORMULATION OF GLP-1 RECEPTOR AGONISTS

(71) Applicants: Matthew L. Zierhut, San Diego, CA (US); Brenda B. Cirincione, San Diego, CA (US)

(72) Inventors: Matthew L. Zierhut, San Diego, CA (US); Brenda B. Cirincione, San Diego, CA (US)

(73) Assignees: Astrazeneca Pharamceuticals LP, WIlmington, DE (US); Amylin Pharmaceuticals LLC, San Diego, CA (US)

(21) Appl. No.: 14/127,364

(22) PCT Filed: Jun. 21, 2012
(86) PCT No.: PCT/US12/43615

§ 371 Date: Mar. 19, 2014

Related U.S. Application Data
(60) Provisional application No. 61/501,018, filed on Jun. 24, 2011.
(60) Provisional application No. 61/657,595, filed on Jun. 8, 2012.

(51) Int. Cl. A61K 9/16; A61K 38/26

(52) U.S. Cl. CPC A61K 9/1647; A61K 38/26
USPC 424/489; 514/11.7

(57) ABSTRACT

The disclosure provides methods for treating diabetes, treating overweight, treating obesity, reducing body weight, treating cardiovascular diseases, treating fatty liver diseases, treating gastrointestinal diseases, and treating neurodegenerative diseases through the once monthly administration of pharmaceutical formulations containing a non-aqueous carrier and GLP-1 receptor agonists that provides therapeutically effective plasma concentration levels of the GLP-1 receptor agonists over the course of a month.

### CROSS-REFERENCE TO RELATED APPLICATIONS

This application claims benefit of U.S. provisional patent application No. 61/501,018, filed Jun. 24, 2011 and 61/657,595 filed Jun. 8, 2012, the entire contents of which are incorporated herein for all purposes.

### SEQUENCE LISTING

The instant application contains a Sequence Listing which has been submitted in ASCII format via EFS-Web and is hereby incorporated by reference in its entirety. Said ASCII copy, created on Jun. 21, 2012, is named 2102WO.txt and is 38,753 bytes in size.

### BACKGROUND

Injectable sustained release formulations offer the opportunity to provide therapeutic amounts of active pharmaceutical ingredients over an extended period of time from a single injection, thus eliminating the need for once or twice daily injections. Presently available injectable sustained release formulations utilizing, for example, microspheres and an aqueous carrier, carry several disadvantages. The formulations do not offer long term stability in the aqueous carrier, thus necessitating separate packaging and storage for the microspheres and aqueous carrier, and the patient must take several steps to combine the microspheres and aqueous carrier before administering the injection.

There is a need for formulations and methods of safely administering sustained release pharmaceutical formulations to patients so that the active ingredient will be released in vivo over an extended period of time and without an unacceptable initial burst release. Ideally the active ingredient is released so as to maintain levels within the therapeutic window, i.e., in the concentration range above that needed to cause the desired clinical effect, but below that where undesirable side effects outweigh the benefits of the drug. It is also necessary that this active pharmaceutical ingredient be provided in a manner that is easy and convenient for the patient to self-administer and that is provided in a formulation that maintains stability for a long period of time in a liquid state. The disclosure is directed to these as well as other important ends.

### SUMMARY

The disclosure provides methods for treating diabetes (e.g., Type 1, Type 2, gestational); treating overweight; treating obesity; reducing body weight; treating cardiovascular diseases; treating fatty liver diseases; (e.g., nonalcoholic fatty liver disease (NAFLD); nonalcoholic steatohepatitis (NASH)); treating gastrointestinal diseases; or treating neurodegenerative diseases in patients (e.g., humans) by once monthly administration to the patients of pharmaceutical formulations comprising a pharmaceutically acceptable non-aqueous carrier and a GLP-1 receptor agonist that is present in an amount of 3 mg to 12.5 mg to treat diabetes, treat overweight, treat obesity, reduce body weight, treat the cardiovascular disease, treat fatty liver disease, treat gastrointestinal diseases, or treating neurodegenerative diseases in the patients (e.g., humans). In one embodiment, the GLP-1 receptor agonist is present in an amount of 4 mg to less than 7.5 mg; or from 5 mg to 7 mg; or from 5 mg to 6 mg. In a preferred embodiment, the GLP-1 receptor agonist, preferably exendin-4, is present in an amount of 6 mg to 10 mg, 7 mg to 9 mg, or preferably about 7 mg, about 8 mg, or about 9 mg. The pharmaceutical formulation may be any formulation described herein or described in WO 2010/028257, the disclosure of which is incorporated by reference herein. The GLP-1 receptor agonist may be any known in the art or described herein. In one embodiment, the GLP-1 receptor agonist is exendin-4 or an exendin-4 analog.

The disclosure provides methods for treating diabetes (e.g., Type 1, Type 2, gestational); treating overweight; treating obesity; reducing body weight; treating cardiovascular diseases; treating fatty liver diseases; (e.g., nonalcoholic fatty liver disease (NAFLD); nonalcoholic steatohepatitis (NASH)); treating gastrointestinal diseases; or treating neurodegenerative diseases in patients (e.g., humans) by once monthly administration to the patients of pharmaceutical formulations comprising a pharmaceutically acceptable non-aqueous carrier and a GLP-1 receptor agonist that is present in an amount of 3 mg to 12.5 mg to treat diabetes, treat overweight, treat obesity, reduce body weight, treat the cardiovascular disease, treat fatty liver disease, treat gastrointestinal diseases, or treating neurodegenerative diseases in the patients (e.g., humans). In one embodiment where the formulation comprises from 7.5 mg to 12 mg of a GLP-1 receptor agonist, the once monthly administration of the formulation achieves a therapeutically effective mean steady state plasma concentration of the GLP-1 receptor agonist, preferably exendin-4, of 170 pg/ml to 330 pg/ml for at least one month. In one embodiment where the formulation comprises from 6 mg to 10 mg, 8 mg to 12 mg, 7 mg to 9 mg, or preferably about 7 mg, about 8 mg, or about 9 mg, of the GLP-1 receptor agonist, preferably exendin-4, the once monthly administration of the formulation achieves a therapeutically effective mean steady state plasma concentration of the GLP-1 receptor agonist of 200 pg/ml to 300 pg/ml for at least one month. In one embodiment where the formulation comprises from 4 mg to less than 7.5 mg of a GLP-1 receptor agonist, the once monthly administration of the formulation achieves a therapeutically effective mean steady state plasma concentration of the GLP-1 receptor agonist of 90 pg/ml to 160 pg/ml for at least one month. In one embodiment where the formulation comprises from 4 mg to less than 7.5 mg of the GLP-1 receptor agonist, the once monthly administration of the formulation achieves a therapeutically effective mean steady state plasma concentration of the GLP-1 receptor agonist of 100 pg/ml to 150 pg/ml for at least one month. In one embodiment where the formulation comprises from 4 mg to less than 7.5 mg of the GLP-1 receptor agonist, the once monthly administration of the formulation achieves a therapeutically effective mean steady state plasma concentration of the GLP-1 receptor agonist of 105 pg/ml to 145 pg/ml for at least one month. In one embodiment where the formulation comprises from 4 mg to less than 7.5 mg of the GLP-1 receptor agonist, the once monthly administration of the formulation achieves a therapeutically effective mean steady state plasma concentration of the GLP-1 receptor agonist of 110 pg/ml to 140 pg/ml for at least one month. In one embodiment where the formulation comprises 4 mg to less than 7.5 mg of the GLP-1 receptor agonist, the once monthly administration of the formulation achieves a therapeutically effective mean steady state plasma concentration of the GLP-1 receptor agonist of 115 pg/ml to 135 pg/ml for at least one month. The pharmaceutical formulation may be any formulation described herein or described in WO 2010/028257, the disclosure of which is incorporated by reference herein. The GLP-1 receptor agonist may be any known in the art or described herein. In one embodiment, the GLP-1 receptor agonist is exendin-4 or an exendin-4 analog. The GLP-1 receptor agonist may be any known in the art or described herein. In one embodiment, the GLP-1 receptor agonist is exenatide.

### BRIEF DESCRIPTION OF THE DRAWINGS

For each of FIGS. 1-6, the microspheres comprise a poly(lactide-co-glycolide) copolymer having exenatide dispersed therein, as described in Example 1. For each of FIGS. 2-6, the oil carrier is a medium chain triglyceride (MCT) commercially available as MIGLYOL® 812 (Sasol Germany GmbH, Witten, Germany).

FIG. 1 provides a comparison of the pharmacokinetics of four different formulations of microspheres. In three formulations, the carrier was an oil (e.g., sesame oil; MIGLYOL® 812; ethyl oleate). In the comparative formulation, the carrier was an aqueous diluent.

FIG. 2 is a graphical simulation (i.e., nanoparametric superposition) of data extrapolated from FIG. 1 of the plasma exenatide concentration over time for the microsphere formulation comprising the oil carrier and the microsphere formulation comprising the aqueous carrier in male Sprague Dawley Rats. The plasma concentration plateau of exenatide may be reached after about 5 dosings.

FIG. 3 illustrates the in vitro release for a formulation comprising microspheres in an oil carrier compared to formulations comprising microspheres in an aqueous carrier.

FIG. 4 illustrates the in vivo release profile in rats over 10 hours for a formulation comprising microspheres in an oil carrier and a formulation comprising microspheres in an aqueous carrier.

FIGS. 5A and B illustrate the purity of exenatide over 9 months at temperatures of 5° C. and 6 months at 25° C. when stored in the formulations comprising the microspheres of Example 1 with an oil carrier as compared to the purity of exenatide that was stored in dry microspheres of Example 1. In FIG. 5A, the purity of exenatide was determined by strong cation exchange HPLC. In FIG. 5B, the purity of exenatide was determined by reverse-phase HPLC.

FIG. 6 illustrates the stability/potency of exenatide in a formulation where the microspheres are suspended in an oil carrier, where one formulation is stored at 5° C. and one formulation is stored at 25° C.

FIG. 7 shows the blinded, randomized, controlled, feasibility study design described in Example 10.

FIG. 8 shows the results of the study described in Example 10. Included within the table are the number of patients in each study arm; the baseline A1C for each study arm; the change from baseline in A1C after 20 weeks of treatment; the percentage of patients in each treatment arm who had an A1C of less than 7% after 20 weeks of treatment; the change from baseline in fasting plasma glucose (FPG) after 20 weeks of treatment; and the change from baseline in weight after 20 weeks of treatment.

FIG. 9 shows the plasma exenatide concentration (pg/mL) over a period of 24 weeks for ExQM 5 mg administered at each of Weeks 0, 4, 8, 12, 16, and 20. The mean steady state concentration for ExQM 5 mg was 127 pg/ml.

FIG. 10 shows the plasma exenatide concentration (pg/mL) over a period of 24 weeks for ExQM 8 mg administered at each of Weeks 0, 4, 8, 12, 16, and 20. The mean steady state concentration for ExQM 8 mg was 247 pg/ml.

FIG. 11 shows the plasma exenatide concentration (pg/mL) over a period of 24 weeks for ExQM 11 mg administered at each of Weeks 0, 4, 8, 12, 16, and 20. The mean steady state concentration for ExQM 11 mg was 218 pg/ml.

FIG. 12 is a graph showing that the in vitro exenatide release for the exenatide suspension accurately predicts the actual in vivo exenatide release of a single 10 mg dose.

FIG. 13 is another depiction of the in vivo exenatide release for a 10 mg dose of the exenatide suspension; and a magnified view of the first 8 hours after injection.

FIG. 14 is a graph illustrating the same data as shown in the graph of FIG. 12 except that it reports cumulative release percent of the single 10 mg dose of exenatide suspension.

FIG. 15 shows the predicted percent of subjects achieving HbA1c reduction versus dose of exenatide once monthly suspension between weeks 24 and 28.

FIG. 16 shows the predicted percentages of subjects achieving a Cₐᵥₑ threshold between weeks 24 and 28

FIG. 17 shows the percentage of subjects with plasma exenatide concentrations above 200 pg/mL between weeks 24 and 28 stratified by the duration of dosing interval spent above the target.

### DETAILED DESCRIPTION

The disclosure provides method for treating diabetes in patients (e.g., humans) by administering to the patients pharmaceutical formulations comprising (i) a pharmaceutically acceptable non-aqueous carrier comprising one or more triglycerides of C₆-C₁₂ fatty acids; and (ii) microspheres comprising a poly(lactide-co-glycolide) polymer having dispersed therein about 5% (w/w) exenatide; and about 2% (w/w) sucrose; wherein the ratio of lactide:glycolide in the polymer is about 1:1; and wherein the exenatide is present in an amount of 3 mg to 12 mg; to treat diabetes in the patients. In one embodiment, the pharmaceutical formulation is administered to the patient once per month or once every four weeks. In one embodiment, the exenatide is present in an amount of 7.5 mg to 12 mg. In one embodiment, the exenatide is present in an amount of 8 mg to 12 mg. In one embodiment, the exenatide is present in an amount of 4 mg to 12 mg. In one embodiment, the exenatide is present in an amount of 4 mg to 11 mg. In one embodiment, the exenatide is present in an amount of 4 mg to 10 mg. In one embodiment, the exenatide is present in an amount of 4 mg to 9 mg. In a preferred embodiment the exenatide is present in an amount of 6 mg to 10 mg, 7 mg to 9 mg, or about 7 mg, about 8 mg, or about 9 mg.

In one embodiment, the exenatide is present in an amount of 4 mg to 8 mg. In one embodiment, the exenatide is present in an amount of 4 mg to less than 7.5 mg. In one embodiment, the exenatide is present in an amount of 4 mg to 7 mg. In one embodiment, the exenatide is present in an amount of 4 mg to 6 mg. In one embodiment, the exenatide is present in an amount of 5 mg to 12 mg. In one embodiment, the exenatide is present in an amount of 5 mg to 11 mg. In one embodiment, the exenatide is present in an amount of 5 mg to 10 mg. In one embodiment, the exenatide is present in an amount of 5 mg to 9 mg. In one embodiment, the exenatide is present in an amount of 5 mg to 8 mg. In one embodiment, the exenatide is present in an amount of 5 mg to less than 7.5 mg. In a preferred embodiment the exenatide is present in an amount of 6 mg to 10 mg, 7 mg to 9 mg, or about 7 mg, 8 mg, or about 9 mg.

In one embodiment, the exenatide is present in an amount of 5 mg to 7 mg. In one embodiment, the exenatide is present in an amount of 5 mg to 6 mg. The diabetes can be Type 1 diabetes, Type 2 diabetes, or gestational diabetes. In one embodiment, the diabetes is Type 1 diabetes.

The disclosure provides method for treating diabetes in patients (e.g., humans) by administering to patients pharmaceutical formulations comprising (i) a pharmaceutically acceptable non-aqueous carrier comprising one or more triglycerides of C₆-C₁₂ fatty acids; and (ii) microspheres comprising a poly(lactide-co-glycolide) polymer having dispersed therein about 5% (w/w) exenatide; and about 2% (w/w) sucrose; wherein the ratio of lactide:glycolide in the polymer is about 1:1; and wherein the exenatide is present in an amount of 8 mg to 12 mg; to treat diabetes in the patients. In one embodiment, the pharmaceutical formulation is administered to the patient once per month or once every four weeks. In one embodiment where the formulation comprises from 8 mg to 12 mg exenatide, from 6 mg to 10 mg, 7 mg to 9 mg, or preferably about 7 mg, 8 mg, or about 9 mg, the once monthly administration of the formulation achieves a therapeutically effective mean steady state plasma concentration of exenatide of 170 pg/ml to 330 pg/ml for at least one month. In one embodiment where the formulation comprises from 8 mg to 12 mg exenatide, the once monthly administration of the formulation achieves a therapeutically effective mean steady state plasma concentration of exenatide of 200 pg/ml to 300 pg/ml for at least one month. The mean steady state plasma concentration is generally measured at least after administration of 2 doses, preferably after administration of 3 doses of the pharmaceutical formulation.

The disclosure provides method for treating diabetes in patients (e.g., humans) by administering to the patients pharmaceutical formulations comprising (i) a pharmaceutically acceptable non-aqueous carrier comprising one or more triglycerides of C₆-C₁₂ fatty acids; and (ii) microspheres comprising a poly(lactide-co-glycolide) polymer having dispersed therein about 5% (w/w) exenatide; and about 2% (w/w) sucrose; wherein the ratio of lactide:glycolide in the polymer is about 1:1; and wherein the exenatide is present in an amount of 5 mg to 7 mg; to treat diabetes in the patients. In one embodiment, the pharmaceutical formulation is administered to the patient once per month or once every four weeks. In one embodiment where the formulation comprises from 5 mg to 7 mg exenatide, the once monthly administration of the formulation achieves a therapeutically effective mean steady state plasma concentration of exenatide of 90 pg/ml to 160 pg/ml for at least one month. In one embodiment where the formulation comprises from 5 mg to 7 mg exenatide, the once monthly administration of the formulation achieves a therapeutically effective mean steady state plasma concentration of exenatide of 100 pg/ml to 150 pg/ml for at least one month. In one embodiment where the formulation comprises from 5 mg to 7 mg exenatide, the once monthly administration of the formulation achieves a therapeutically effective mean steady state plasma concentration of exenatide of 105 pg/ml to 145 pg/ml for at least one month. In one embodiment where the formulation comprises from 5 mg to 7 mg exenatide, the once monthly administration of the formulation achieves a therapeutically effective mean steady state plasma concentration of exenatide of 110 pg/ml to 140 pg/ml for at least one month. In one embodiment where the formulation comprises from 5 mg to 7 mg exenatide, the once monthly administration of the formulation achieves a therapeutically effective mean steady state plasma concentration of exenatide of 115 pg/ml to 135 pg/ml for at least one month. The mean steady state plasma concentration is generally measured at least after administration of 2 doses, preferably after administration of 3 doses of the pharmaceutical formulation.

The disclosure provides method for treating diabetes in patients (e.g., humans) by administering to the patients pharmaceutical formulations comprising (i) a pharmaceutically acceptable non-aqueous carrier comprising one or more triglycerides of C₆-C₁₂ fatty acids; and (ii) microspheres comprising a poly(lactide-co-glycolide) polymer having dispersed therein about 5% (w/w) exenatide; and about 2% (w/w) sucrose; wherein the ratio of lactide:glycolide in the polymer is about 1:1; and wherein the exenatide is present in an amount of 5 mg to 6 mg; to treat diabetes in the patients. In one embodiment, the pharmaceutical formulation is administered to the patient once per month or once every four weeks. In one embodiment where the formulation comprises from 5 mg to 6 mg exenatide, the once monthly administration of the formulation achieves a therapeutically effective mean steady state plasma concentration of exenatide of 90 pg/ml to 160 pg/ml for at least one month. In one embodiment where the formulation comprises from 5 mg to 6 mg exenatide, the once monthly administration of the formulation achieves a therapeutically effective mean steady state plasma concentration of exenatide of 100 pg/ml to 150 pg/ml for at least one month. In one embodiment where the formulation comprises from 5 mg to 6 mg exenatide, the once monthly administration of the formulation achieves a therapeutically effective mean steady state plasma concentration of exenatide of 105 pg/ml to 145 pg/ml for at least one month. In one embodiment where the formulation comprises from 5 mg to 6 mg exenatide, the once monthly administration of the formulation achieves a therapeutically effective mean steady state plasma concentration of exenatide of 110 pg/ml to 140 pg/ml for at least one month. In one embodiment where the formulation comprises from 5 mg to 6 mg exenatide, the once monthly administration of the formulation achieves a therapeutically effective mean steady state plasma concentration of exenatide of 115 pg/ml to 135 pg/ml for at least one month. The mean steady state plasma concentration is generally measured at least after administration of 2 doses, preferably after administration of 3 doses of the pharmaceutical formulation.

The disclosure provides methods for treating overweight; treating obesity; reducing body weight; treating cardiovascular diseases; treating fatty liver diseases; (e.g., nonalcoholic fatty liver disease (NAFLD); nonalcoholic steatohepatitis (NASH)); treating gastrointestinal diseases; or treating neurodegenerative diseases in patients (e.g., humans) by administering to the patients pharmaceutical formulations comprising (i) a pharmaceutically acceptable non-aqueous carrier comprising one or more triglycerides of C₆-C₁₂ fatty acids; and (ii) microspheres comprising a poly(lactide-co-glycolide) polymer having dispersed therein about 5% (w/w) exenatide; and about 2% (w/w) sucrose; wherein the ratio of lactide:glycolide in the polymer is about 1:1; and wherein the exenatide is present in an amount of 3 mg to 12 mg, 6 mg to 10 mg, 7 mg, to 9 mg, or preferably about 7 mg, 8 mg, or about 9 mg; to treat overweight, treat obesity, reduce body weight, treat a cardiovascular disease, treat fatty liver disease (e.g., nonalcoholic fatty liver disease (NAFLD); nonalcoholic steatohepatitis (NASH)); treat a gastrointestinal disease, or treat a neurodegenerative disease in the patients (e.g., humans). In one embodiment, the pharmaceutical formulation is administered to the patient once per month or once every four weeks. In one embodiment, the pharmaceutical formulation is administered to the patient once per month or once every four weeks. In one embodiment, the exenatide is present in an amount of 7.5 mg to 12 mg. In one embodiment, the exenatide is present in an amount of 8 mg to 12 mg. In one embodiment, the exenatide is present in an amount of 4 mg to 12 mg, 6 mg to 10 mg, 7 mg to 9 mg, or preferably about 7 mg, about 8 mg, or about 9 mg. In one embodiment, the exenatide is present in an amount of 4 mg to 11 mg. In one embodiment, the exenatide is present in an amount of 4 mg to 10 mg. In one embodiment, the exenatide is present in an amount of 4 mg to 9 mg. In one embodiment, the exenatide is present in an amount of 4 mg to 8 mg. In one embodiment, the exenatide is present in an amount of 4 mg to less than 7.5 mg. In one embodiment, the exenatide is present in an amount of 4 mg to 7 mg. In one embodiment, the exenatide is present in an amount of 4 mg to 6 mg. In one embodiment, the exenatide is present in an amount of 5 mg to 12 mg. In one embodiment, the exenatide is present in an amount of 5 mg to 11 mg. In one embodiment, the exenatide is present in an amount of 5 mg to 10 mg. In one embodiment, the exenatide is present in an amount of 5 mg to 9 mg. In one embodiment, the exenatide is present in an amount of 5 mg to 8 mg. In one embodiment, the exenatide is present in an amount of 5 mg to less than 7.5 mg. In one embodiment, the exenatide is present in an amount of 5 mg to 7 mg. In one embodiment, the exenatide is present in an amount of 5 mg to 6 mg. In a preferred embodiment the exenatide is present in an amount of 6 mg to 10 mg, 7 mg to 9 mg, or about 7 mg, 8 mg, or about 9 mg. In one embodiment, the method is for treating overweight. In one embodiment, the method is for treating obesity. In one embodiment, the method is for reducing body weight.

The disclosure provides methods for treating overweight; treating obesity; reducing body weight; treating cardiovascular diseases; treating fatty liver diseases; (e.g., nonalcoholic fatty liver disease (NAFLD); nonalcoholic steatohepatitis (NASH)); treating gastrointestinal diseases; or treating neurodegenerative diseases in patients (e.g., humans) by administering to the patients pharmaceutical formulations comprising (i) a pharmaceutically acceptable non-aqueous carrier comprising one or more triglycerides of C₆-C₁₂ fatty acids; and (ii) microspheres comprising a poly(lactide-co-glycolide) polymer having dispersed therein about 5% (w/w) exenatide; and about 2% (w/w) sucrose; wherein the ratio of lactide:glycolide in the polymer is about 1:1; and wherein the exenatide is present in an amount of 8 mg to 12 mg, 6 mg to 10 mg, 7 mg to 9 mg, or preferably about 7 mg, about 8 mg, or about 9 mg; to treat overweight, treat obesity, reduce body weight, treat a cardiovascular disease, treat fatty liver disease (e.g., nonalcoholic fatty liver disease (NAFLD); nonalcoholic steatohepatitis (NASH)); treat a gastrointestinal disease, or treat a neurodegenerative disease in the patients (e.g., humans). In one embodiment, the pharmaceutical formulation is administered to the patient once per month or once every four weeks. In one embodiment where the formulation comprises from 8 mg to 12 mg exenatide, 6 mg to 10 mg, 7 mg to 9 mg, or preferably about 7 mg, about 8 mg, or about 9 mg, the once monthly administration of the formulation achieves a therapeutically effective mean steady state plasma concentration of exenatide of 170 pg/ml to 330 pg/ml for at least one month. In one embodiment where the formulation comprises from 8.0 mg to 12 mg exenatide, the once monthly administration of the formulation achieves a therapeutically effective mean steady state plasma concentration of exenatide of 200 pg/ml to 300 pg/ml for at least one month. The mean steady state plasma concentration is generally measured at least after administration of 2 doses, preferably after administration of 3 doses of the pharmaceutical formulation. In one embodiment, the method is for treating overweight. In one embodiment, the method is for treating obesity. In one embodiment, the method is for reducing body weight.

The disclosure provides methods for treating overweight; treating obesity; reducing body weight; treating cardiovascular diseases; treating fatty liver diseases; (e.g., nonalcoholic fatty liver disease (NAFLD); nonalcoholic steatohepatitis (NASH)); treating gastrointestinal diseases; or treating neurodegenerative diseases in patients (e.g., humans) by administering to the patients pharmaceutical formulations comprising (i) a pharmaceutically acceptable non-aqueous carrier comprising one or more triglycerides of C₆-C₁₂ fatty acids; and (ii) microspheres comprising a poly(lactide-co-glycolide) polymer having dispersed therein about 5% (w/w) exenatide; and about 2% (w/w) sucrose; wherein the ratio of lactide:glycolide in the polymer is about 1:1; and wherein the exenatide is present in an amount of 5 mg to 7 mg; to treat overweight, treat obesity, reduce body weight, treat a cardiovascular disease, treat fatty liver disease (e.g., nonalcoholic fatty liver disease (NAFLD); nonalcoholic steatohepatitis (NASH)); treat a gastrointestinal disease, or treat a neurodegenerative disease in the patients (e.g., humans). In one embodiment, the pharmaceutical formulation is administered to the patient once per month or once every four weeks. In one embodiment where the formulation comprises from 5 mg to 7 mg exenatide, the once monthly administration of the formulation achieves a therapeutically effective mean steady state plasma concentration of exenatide of 90 pg/ml to 160 pg/ml for at least one month. In one embodiment where the formulation comprises from 5 mg to 7 mg exenatide, the once monthly administration of the formulation achieves a therapeutically effective mean steady state plasma concentration of exenatide of 100 pg/ml to 150 pg/ml for at least one month. In one embodiment where the formulation comprises from 5 mg to 7 mg exenatide, the once monthly administration of the formulation achieves a therapeutically effective mean steady state plasma concentration of exenatide of 105 pg/ml to 145 pg/ml for at least one month. In one embodiment where the formulation comprises from 5 mg to 7 mg exenatide, the once monthly administration of the formulation achieves a therapeutically effective mean steady state plasma concentration of exenatide of 110 pg/ml to 140 pg/ml for at least one month. In one embodiment where the formulation comprises from 5 mg to 7 mg exenatide, the once monthly administration of the formulation achieves a therapeutically effective mean steady state plasma concentration of exenatide of 115 pg/ml to 135 pg/ml for at least one month. The mean steady state plasma concentration is generally measured at least after administration of 2 doses, preferably after administration of 3 doses of the pharmaceutical formulation. In one embodiment, the method is for treating overweight. In one embodiment, the method is for treating obesity. In one embodiment, the method is for reducing body weight.

The disclosure provides methods for treating overweight; treating obesity; reducing body weight; treating cardiovascular diseases; treating fatty liver diseases; (e.g., nonalcoholic fatty liver disease (NAFLD); nonalcoholic steatohepatitis (NASH)); treating gastrointestinal diseases; or treating neurodegenerative diseases in patients (e.g., humans) by administering to the patients pharmaceutical formulations comprising (i) a pharmaceutically acceptable non-aqueous carrier comprising one or more triglycerides of C₆-C₁₂ fatty acids; and (ii) microspheres comprising a poly(lactide-co-glycolide) polymer having dispersed therein about 5% (w/w) exenatide; and about 2% (w/w) sucrose; wherein the ratio of lactide:glycolide in the polymer is about 1:1; and wherein the exenatide is present in an amount of 5 mg to 6 mg; to treat overweight, treat obesity, reduce body weight, treat a cardiovascular disease, treat fatty liver disease (e.g., nonalcoholic fatty liver disease (NAFLD); nonalcoholic steatohepatitis (NASH)); treat a gastrointestinal disease, or treat a neurodegenerative disease in the patients (e.g., humans). In one embodiment, the pharmaceutical formulation is administered to the patient once per month or once every four weeks. In one embodiment where the formulation comprises from 5 mg to 6 mg exenatide, the once monthly administration of the formulation achieves a therapeutically effective mean steady state plasma concentration of exenatide of 90 pg/ml to 160 pg/ml for at least one month. In one embodiment where the formulation comprises from 5 mg to 6 mg exenatide, the once monthly administration of the formulation achieves a therapeutically effective mean steady state plasma concentration of exenatide of 100 pg/ml to 150 pg/ml for at least one month. In one embodiment where the formulation comprises from 5 mg to 6 mg exenatide, the once monthly administration of the formulation achieves a therapeutically effective mean steady state plasma concentration of exenatide of 105 pg/ml to 145 pg/ml for at least one month. In one embodiment where the formulation comprises from 5 mg to 6 mg exenatide, the once monthly administration of the formulation achieves a therapeutically effective mean steady state plasma concentration of exenatide of 110 pg/ml to 140 pg/ml for at least one month. In one embodiment where the formulation comprises from 5 mg to 6 mg exenatide, the once monthly administration of the formulation achieves a therapeutically effective mean steady state plasma concentration of exenatide of 115 pg/ml to 135 pg/ml for at least one month. The mean steady state plasma concentration is generally measured at least after administration of 2 doses, preferably after administration of 3 doses of the pharmaceutical formulation. In one embodiment, the method is for treating overweight. In one embodiment, the method is for treating obesity. In one embodiment, the method is for reducing body weight.

The disclosure provides method for treating diabetes in patients (e.g., humans) by administering to the patients (e.g., humans) pharmaceutical formulations comprising (i) a pharmaceutically acceptable non-aqueous carrier; and (ii) microspheres which comprise a biocompatible, biodegradable polymer and a GLP-1 receptor agonist; wherein the GLP-1 receptor agonist, preferably exendin-4, is present in an amount of 3 mg to 12 mg, 6 mg to 10 mg, 7 mg to 9 mg, or preferably about 7 mg, about 8 mg, or about 9 mg; to treat diabetes in the patients (e.g., humans). In one embodiment, the pharmaceutical formulation is administered to the patient once per month or once every four weeks. In one embodiment, the exenatide is present in an amount of 7.5 mg to 12 mg. In one embodiment, the GLP-1 receptor agonist is present in an amount of 8 mg to 12 mg. In one embodiment, the GLP-1 receptor agonist is present in an amount of 4 mg to 12 mg. In one embodiment, the GLP-1 receptor agonist is present in an amount of 4 mg to 11 mg. In one embodiment, the GLP-1 receptor agonist is present in an amount of 4 mg to 10 mg. In one embodiment, the GLP-1 receptor agonist is present in an amount of 4 mg to 9 mg. In one embodiment, the GLP-1 receptor agonist is present in an amount of 4 mg to 8 mg. In one embodiment, the GLP-1 receptor agonist is present in an amount of 4 mg to less than 7.5 mg. In one embodiment, the GLP-1 receptor agonist is present in an amount of 4 mg to 7 mg. In one embodiment, the GLP-1 receptor agonist is present in an amount of 4 mg to 6 mg. In one embodiment, the GLP-1 receptor agonist is present in an amount of 5 mg to 12 mg. In one embodiment, the GLP-1 receptor agonist is present in an amount of 5 mg to 11 mg. In one embodiment, the GLP-1 receptor agonist is present in an amount of 5 mg to 10 mg. In one embodiment, the GLP-1 receptor agonist is present in an amount of 5 mg to 9 mg. In one embodiment, the GLP-1 receptor agonist is present in an amount of 5 mg to 8 mg. In one embodiment, the GLP-1 receptor agonist is present in an amount of 5 mg to less than 7.5 mg. In one embodiment, the GLP-1 receptor agonist is present in an amount of 5 mg to 7 mg. In one embodiment, the GLP-1 receptor agonist is present in an amount of 5 mg to 6 mg. The GLP-1 receptor agonist can be any known in the art, including those described herein. In one embodiment, the GLP-1 receptor agonist is exenatide. The pharmaceutical formulation may be any described herein or in WO 2010/028257, the disclosure of which is incorporated herein by reference. The diabetes can be Type 1 diabetes, Type 2 diabetes, or gestational diabetes. In one embodiment, the diabetes is Type 1 diabetes. In a preferred embodiment the exenatide is present in an amount of 6 mg to 10 mg, 7 mg to 9 mg, or about 7 mg, about 8 mg, or about 9 mg.

The disclosure provides method for treating diabetes in patients (e.g., humans) by administering to patients pharmaceutical formulations comprising (i) a pharmaceutically acceptable non-aqueous carrier; and (ii) microspheres which comprise a biocompatible, biodegradable polymer and a GLP-1 receptor agonist; wherein the GLP-1 receptor agonist is present in an amount of 8 mg to 12 mg, of 6 mg to 10 mg, 7 mg to 9 mg, or preferably about 7 mg, about 8 mg, or about 9 mg; to treat diabetes in the patients. In one embodiment, the pharmaceutical formulation is administered to the patient once per month or once every four weeks. In one embodiment where the formulation comprises from 8 mg to 12 mg, 6 mg to 10 mg, 7 mg to 9 mg, or preferably about 7 mg, 8 mg, or about 9 mg of the GLP-1 receptor agonist, the once monthly administration of the formulation achieves a therapeutically effective mean steady state plasma concentration of the GLP-1 receptor agonist of 170 pg/ml to 330 pg/ml for at least one month. In one embodiment where the formulation comprises from 8 mg to 12 mg of the GLP-1 receptor agonist, the once monthly administration of the formulation achieves a therapeutically effective mean steady state plasma concentration of the GLP-1 receptor agonist of 200 pg/ml to 300 pg/ml for at least one month. The mean steady state plasma concentration is generally measured at least after administration of 2 doses, preferably after administration of 3 doses of the pharmaceutical formulation. The GLP-1 receptor agonist can be any known in the art, including those described herein. In one embodiment, the GLP-1 receptor agonist is exenatide. The pharmaceutical formulation may be any described herein or in WO 2010/028257, the disclosure of which is incorporated herein by reference. The diabetes can be Type 1 diabetes, Type 2 diabetes, or gestational diabetes. In one embodiment, the diabetes is Type 1 diabetes.

The disclosure provides method for treating diabetes in patients (e.g., humans) by administering to the patients pharmaceutical formulations comprising (i) a pharmaceutically acceptable non-aqueous carrier; and (ii) microspheres which comprise a biocompatible, biodegradable polymer and a GLP-1 receptor agonist; wherein the GLP-1 receptor agonist is present in an amount of 5 mg to 7 mg; to treat diabetes in the patients. In one embodiment, the pharmaceutical formulation is administered to the patient once per month or once every four weeks. In one embodiment where the formulation comprises from 5 mg to 7 mg of the GLP-1 receptor agonist, the once monthly administration of the formulation achieves a therapeutically effective mean steady state plasma concentration of the GLP-1 receptor agonist of 90 pg/ml to 160 pg/ml for at least one month. In one embodiment where the formulation comprises from 5 mg to 7 mg of the GLP-1 receptor agonist, the once monthly administration of the formulation achieves a therapeutically effective mean steady state plasma concentration of the GLP-1 receptor agonist of 100 pg/ml to 150 pg/ml for at least one month. In one embodiment where the formulation comprises from 5 mg to 7 mg the GLP-1 receptor agonist, the once monthly administration of the formulation achieves a therapeutically effective mean steady state plasma concentration of the GLP-1 receptor agonist of 105 pg/ml to 145 pg/ml for at least one month. In one embodiment where the formulation comprises from 5 mg to 7 mg of the GLP-1 receptor agonist, the once monthly administration of the formulation achieves a therapeutically effective mean steady state plasma concentration of the GLP-1 receptor agonist of 110 pg/ml to 140 pg/ml for at least one month. In one embodiment where the formulation comprises from 5 mg to 7 mg of the GLP-1 receptor agonist, the once monthly administration of the formulation achieves a therapeutically effective mean steady state plasma concentration of the GLP-1 receptor agonist of 115 pg/ml to 135 pg/ml for at least one month. The mean steady state plasma concentration is generally measured at least after administration of 2 doses, preferably after administration of 3 doses of the pharmaceutical formulation. The GLP-1 receptor agonist can be any known in the art, including those described herein. In one embodiment, the GLP-1 receptor agonist is exenatide. The pharmaceutical formulation may be any described herein or in WO 2010/028257, the disclosure of which is incorporated herein by reference.

The disclosure provides method for treating diabetes in patients (e.g., humans) by administering to the patients pharmaceutical formulations comprising (i) a pharmaceutically acceptable non-aqueous carrier; and (ii) microspheres which comprise a biocompatible, biodegradable polymer and a GLP-1 receptor agonist; wherein the GLP-1 receptor agonist is present in an amount of 5 mg to 6 mg; to treat diabetes in the patients. In one embodiment, the pharmaceutical formulation is administered to the patient once per month or once every four weeks. In one embodiment where the formulation comprises from 5 mg to 6 mg of the GLP-1 receptor agonist, the once monthly administration of the formulation achieves a therapeutically effective mean steady state plasma concentration of the GLP-1 receptor agonist of 90 pg/ml to 160 pg/ml for at least one month. In one embodiment where the formulation comprises from 5 mg to 6 mg of the GLP-1 receptor agonist, the once monthly administration of the formulation achieves a therapeutically effective mean steady state plasma concentration of the GLP-1 receptor agonist of 100 pg/ml to 150 pg/ml for at least one month. In one embodiment where the formulation comprises from 5 mg to 6 mg the GLP-1 receptor agonist, the once monthly administration of the formulation achieves a therapeutically effective mean steady state plasma concentration of the GLP-1 receptor agonist of 105 pg/ml to 145 pg/ml for at least one month. In one embodiment where the formulation comprises from 5 mg to 6 mg of the GLP-1 receptor agonist, the once monthly administration of the formulation achieves a therapeutically effective mean steady state plasma concentration of the GLP-1 receptor agonist of 110 pg/ml to 140 pg/ml for at least one month. In one embodiment where the formulation comprises from 5 mg to 6 mg of the GLP-1 receptor agonist, the once monthly administration of the formulation achieves a therapeutically effective mean steady state plasma concentration of the GLP-1 receptor agonist of 115 pg/ml to 135 pg/ml for at least one month. The mean steady state plasma concentration is generally measured at least after administration of 2 doses, preferably after administration of 3 doses of the pharmaceutical formulation. The GLP-1 receptor agonist can be any known in the art, including those described herein. In one embodiment, the GLP-1 receptor agonist is exenatide. The pharmaceutical formulation may be any described herein or in WO 2010/028257, the disclosure of which is incorporated herein by reference.

The disclosure provides method for treating diabetes, delaying gastric emptying, and/or treating obesity in patients (e.g., humans) in need thereof by administering to the patients a monthly dose of a pharmaceutical suspension that delivers 3 mg-12 mg, 5 mg-11 mg, 6 mg-10 mg, 7 mg-9 mg, about 7 mg, about 8 mg, or about 9 mg of exenatide or a GLP-1 receptor agonist per dose to a human in need thereof, wherein the pharmaceutical suspension comprises:
(1) a pharmaceutically acceptable non-aqueous carrier having one or more triglycerides of C₆-C₁₂ fatty acids; and
(2) microspheres comprising a poly(lactide-co-glycolide) polymer having dispersed therein about 5% (w/w) exenatide or GLP-1 agonist, and about 2% (w/w) sucrose; wherein the ratio of lactide:glycolide in the polymer is about 1:1;

and wherein the administration of an initial dose of the formulation achieves an in vivo release profile having a small transient rise over the first 8 hours, followed by a plateau, wherein less than 0.5% of the exenatide or GLP1 receptor agonist is released within the first 24 hours;

and wherein the in vivo release profile at steady state has the following characteristics:
(i) the maximum plasma concentration is achieved at approximately 2 weeks after each monthly dose;
(ii) the peak to trough ratio following each monthly dose ranges between 5 to 9, or is about 5, 6, 7, 8, or 9.

In one embodiment, the method the Cₘₐₓ for the exenatide or GLP-1 receptor agonist at steady state is 150 pg/mL-500 pg/mL, 200 pg/mL-500 pg/mL, 250 pg/mL-500 pg/mL, or 255 pg/mL-500 pg/mL. In another embodiment, the monthly dosing attains the following at steady state:
(1) a monthly Cₘₐₓ of at least 200 pg/mL, 225 pg/mL, 250 pg/mL, 275 pg/mL, 300 pg/mL, 325 pg/mL, 350 pg/mL, 375 pg/mL, 400 pg/mL, 450 pg/mL or 500 pg/ml;
(2) a monthly Cₐᵥₑ exenatide or GLP-1 receptor agonist concentration of at least 100 pg/mL, 125 pg/mL, 150 pg/mL, 175 pg/mL, or 200 pg/mL;
(3) a monthly Cₘᵢₙ of at least 25 pg/mL, 50 pg/mL, 75 pg/mL, 100 pg/mL, 125 pg/mL, or 150 pg/mL.

In some instances, the monthly Cₘᵢₙ is about 50 pg/mL and the Cₘₐₓ is about 250 pg/mL to about 500 pg/mL.

The disclosure is also directed to a method for treating diabetes, delaying gastric emptying, and/or treating obesity comprising monthly administration of a pre-mixed pharmaceutical suspension that delivers 6 mg-10 mg of exenatide to a human in need thereof, wherein the pre-mixed pharmaceutical suspension comprises:
(1) a pharmaceutically acceptable non-aqueous carrier having one or more triglycerides of C₆-C₁₂ fatty acids; and
(2) microspheres comprising a poly(lactide-co-glycolide) polymer having dispersed therein about 5% (w/w) exenatide or GLP-1 agonist, and about 2% (w/w) sucrose; wherein the ratio of lactide:glycolide in the polymer is about 1:1;

and wherein the administration of an initial dose of the formulation achieves an in vivo release profile having a small transient rise over the first 8 hours, followed by a plateau, wherein less than 0.5% of the exenatide or GLP1 receptor agonist is released within the first 24 hours;

and wherein the in vivo release profile at steady state has the following characteristics:
(i) the maximum plasma concentration is achieved at approximately 2 weeks after each monthly dose;
(ii) the peak to trough ratio following each monthly dose ranges between 5 to 9, or is about 5, 6, 7, 8, or 9. In one embodiment, the method delivers 3 mg-12 mg, 5 mg-11 mg, 6 mg-10 mg, 7 mg-9 mg, about 7 mg, about 8 mg, or about 9 mg of exenatide per dose.

In one embodiment, the Cₘₐₓ for the exenatide at steady state is 150 pg/mL-500 pg/mL, 200 pg/mL-500 pg/mL, 250 pg/mL-500 pg/mL, or 255 pg/mL-500 pg/mL. In another embodiment, the monthly dosing attains the following at steady state:
(1) a monthly Cₘₐₓ of at least 200 pg/mL, 225 pg/mL, 250 pg/mL, 275 pg/mL, 300 pg/mL, 325 pg/mL, 350 pg/mL, 375 pg/mL, 400 pg/mL, 450 pg/mL or 500 pg/ml;
(2) a monthly Cₐᵥₑ exenatide or GLP-1 receptor agonist concentration of at least 100 pg/mL, 125 pg/mL, 150 pg/mL, 175 pg/mL, or 200 pg/mL;
(3) a monthly Cₘᵢₙ of at least 25 pg/mL, 50 pg/mL, 75 pg/mL, 100 pg/mL, 125 pg/mL, or 150 pg/mL.

In some instances, the monthly Cₘᵢₙ is about 50 pg/mL and the Cₘₐₓ is about 250 pg/mL to about 500 pg/mL.

In some instances, the methods described herein achieve a reduction of HbA1c levels to less than 7%, 6.5%, 6.0%, or 5.5% at steady state. Likewise, in some instances, the methods described herein provide for at least a 5%, 10%, 15%, or 20% delay in gastric emptying.

The instant disclosure also relates to a monthly injectable unit dosage form of a pharmaceutical suspension for treating diabetes that delivers 3 mg-12 mg, 5 mg-11 mg, 6 mg-10 mg, 7 mg-9 mg, about 7 mg, about 8 mg, or about 9 mg of exenatide or a GLP-1 receptor agonist in a single dose, wherein the suspension comprises:
(1) a pharmaceutically acceptable non-aqueous carrier having one or more triglycerides of C₆-C₁₂ fatty acids; and
(2) microspheres comprising a poly(lactide-co-glycolide) polymer having dispersed therein about 5% (w/w) exenatide or GLP-1 receptor agonist, and about 2% (w/w) sucrose; wherein the ratio of lactide:glycolide in the polymer is about 1:1;

and wherein the administration of an initial dose of the formulation achieves an in vivo release profile having a small transient rise over the first 8 hours, followed by a plateau, wherein less than 0.5% of the exenatide or GLP1 receptor agonist is released within the first 24 hours;

and wherein monthly dosing of the suspension achieves an in vivo release profile at steady state having the following characteristics:
(i) the maximum plasma concentration is achieved at approximately 2 weeks after each monthly dose; and
(ii) the peak to trough ratio following each monthly dose ranges between 5 to 9, or is about 5, 6, 7, 8, or 9.

The disclosure provides methods for treating overweight; treating obesity; reducing body weight; treating cardiovascular diseases; treating fatty liver diseases; (e.g., nonalcoholic fatty liver disease (NAFLD); nonalcoholic steatohepatitis (NASH)); treating gastrointestinal diseases; or treating neurodegenerative diseases in patients (e.g., humans) by administering to the patients (e.g., humans) pharmaceutical formulations comprising (i) a pharmaceutically acceptable non-aqueous carrier; and (ii) microspheres which comprise a biocompatible, biodegradable polymer and a GLP-1 receptor agonist; wherein the GLP-1 receptor agonist is present in an amount of 3 mg to 12 mg, 6 mg to 10 mg, 7 mg, 9 mg, or preferably about 7 mg, about 8 mg, or about 9 mg; to treat overweight, treat obesity, reduce body weight, treat a cardiovascular disease, treat fatty liver disease (e.g., nonalcoholic fatty liver disease (NAFLD); nonalcoholic steatohepatitis (NASH)); treat a gastrointestinal disease, or treat a neurodegenerative disease in the patients (e.g., humans). In one embodiment, the pharmaceutical formulation is administered to the patient once per month or once every four weeks. In one embodiment, the exenatide is present in an amount of 7.5 mg to 12 mg. In one embodiment, the GLP-1 receptor agonist is present in an amount of 8 mg to 12 mg. In one embodiment, the GLP-1 receptor agonist is present in an amount of 4 mg to 12 mg. In one embodiment, the GLP-1 receptor agonist is present in an amount of 4 mg to 11 mg. In one embodiment, the GLP-1 receptor agonist is present in an amount of 4 mg to 10 mg. In one embodiment, the GLP-1 receptor agonist is present in an amount of 4 mg to 9 mg. In one embodiment, the GLP-1 receptor agonist is present in an amount of 4 mg to 8 mg. In one embodiment, the GLP-1 receptor agonist is present in an amount of 4 mg to less than 7.5 mg. In one embodiment, the GLP-1 receptor agonist is present in an amount of 4 mg to 7 mg. In one embodiment, the GLP-1 receptor agonist is present in an amount of 4 mg to 6 mg. In one embodiment, the GLP-1 receptor agonist is present in an amount of 5 mg to 12 mg. In one embodiment, the GLP-1 receptor agonist is present in an amount of 5 mg to 11 mg. In one embodiment, the GLP-1 receptor agonist is present in an amount of 5 mg to 10 mg. In one embodiment, the GLP-1 receptor agonist is present in an amount of 5 mg to 9 mg. In one embodiment, the GLP-1 receptor agonist is present in an amount of 5 mg to 8 mg. In one embodiment, the GLP-1 receptor agonist is present in an amount of 5 mg to less than 7.5 mg. In one embodiment, the GLP-1 receptor agonist is present in an amount of 5 mg to 7 mg. In one embodiment, the GLP-1 receptor agonist is present in an amount of 5 mg to 6 mg. In a preferred embodiment the GLP-1 receptor agonist is present in an amount of 8 mg to 10 mg, or about 9 mg.

The GLP-1 receptor agonist can be any known in the art, including those described herein. In one embodiment, the GLP-1 receptor agonist is exenatide. The pharmaceutical formulation may be any described herein or in WO 2010/028257, the disclosure of which is incorporated herein by reference. In one embodiment, the method is for treating overweight. In one embodiment, the method is for treating obesity. In one embodiment, the method is for reducing body weight.

The disclosure provides methods for treating overweight; treating obesity; reducing body weight; treating cardiovascular diseases; treating fatty liver diseases; (e.g., nonalcoholic fatty liver disease (NAFLD); nonalcoholic steatohepatitis (NASH)); treating gastrointestinal diseases; or treating neurodegenerative diseases in patients (e.g., humans) by administering to the patients pharmaceutical formulations comprising (i) a pharmaceutically acceptable non-aqueous carrier; and (ii) microspheres which comprise a biocompatible, biodegradable polymer and a GLP-1 receptor agonist; wherein the GLP-1 receptor agonist is present in an amount of 8 mg to 12 mg; to treat overweight, treat obesity, reduce body weight, treat a cardiovascular disease, treat fatty liver disease (e.g., nonalcoholic fatty liver disease (NAFLD); nonalcoholic steatohepatitis (NASH)); treat a gastrointestinal disease, or treat a neurodegenerative disease in the patients (e.g., humans). In one embodiment, the pharmaceutical formulation is administered to the patient once per month or once every four weeks. In one embodiment where the formulation comprises from 8 mg to 12 mg, 6 mg to 10 mg, 7 mg to 9 mg, or preferably about 7 mg, 8 mg, or about 9 mg of the GLP-1 receptor agonist, the once monthly administration of the formulation achieves a therapeutically effective mean steady state plasma concentration of the GLP-1 receptor agonist of 170 pg/ml to 330 pg/ml for at least one month. In one embodiment where the formulation comprises from 8 mg to 12 mg, 6 mg to 10 mg, 7 mg to 9 mg, or preferably about 7 mg, about 8 mg, or about 9 mg of the GLP-1 receptor agonist, the once monthly administration of the formulation achieves a therapeutically effective mean steady state plasma concentration of the GLP-1 receptor agonist of 200 pg/ml to 300 pg/ml for at least one month. The GLP-1 receptor agonist can be any known in the art, including those described herein. In one embodiment, the GLP-1 receptor agonist is exenatide. The pharmaceutical formulation may be any described herein or in WO 2010/028257, the disclosure of which is incorporated herein by reference. In one embodiment, the method is for treating overweight. In one embodiment, the method is for treating obesity. In one embodiment, the method is for reducing body weight.

The disclosure provides methods for treating overweight; treating obesity; reducing body weight; treating cardiovascular diseases; treating fatty liver diseases; (e.g., nonalcoholic fatty liver disease (NAFLD); nonalcoholic steatohepatitis (NASH)); treating gastrointestinal diseases; or treating neurodegenerative diseases in patients (e.g., humans) by administering to the patients pharmaceutical formulations comprising (i) a pharmaceutically acceptable non-aqueous carrier; and (ii) microspheres which comprise a biocompatible, biodegradable polymer and a GLP-1 receptor agonist; wherein the GLP-1 receptor agonist is present in an amount of 5 mg to 7 mg; to treat overweight, treat obesity, reduce body weight, treat a cardiovascular disease, treat fatty liver disease (e.g., nonalcoholic fatty liver disease (NAFLD); nonalcoholic steatohepatitis (NASH)); treat a gastrointestinal disease, or treat a neurodegenerative disease in the patients (e.g., humans). In one embodiment, the pharmaceutical formulation is administered to the patient once per month or once every four weeks. In one embodiment where the formulation comprises from 5 mg to 7 mg of the GLP-1 receptor agonist, the once monthly administration of the formulation achieves a therapeutically effective mean steady state plasma concentration GLP-1 receptor agonist of 90 pg/ml to 160 pg/ml for at least one month. In one embodiment where the formulation comprises from 5 mg to 7 mg GLP-1 receptor agonist, the once monthly administration of the formulation achieves a therapeutically effective mean steady state plasma concentration GLP-1 receptor agonist of 100 pg/ml to 150 pg/ml for at least one month. In one embodiment where the formulation comprises from 5 mg to 7 mg GLP-1 receptor agonist, the once monthly administration of the formulation achieves a therapeutically effective mean steady state plasma concentration GLP-1 receptor agonist of 105 pg/ml to 145 pg/ml for at least one month. In one embodiment where the formulation comprises from 5 mg to 7 mg GLP-1 receptor agonist, the once monthly administration of the formulation achieves a therapeutically effective mean steady state plasma concentration GLP-1 receptor agonist of 110 pg/ml to 140 pg/ml for at least one month. In one embodiment where the formulation comprises from 5 mg to 7 mg GLP-1 receptor agonist, the once monthly administration of the formulation achieves a therapeutically effective mean steady state plasma concentration GLP-1 receptor agonist of 115 pg/ml to 135 pg/ml for at least one month. The mean steady state plasma concentration is generally measured at least after administration of 2 doses, preferably after administration of 3 doses of the pharmaceutical formulation. The GLP-1 receptor agonist can be any known in the art, including those described herein. In one embodiment, the GLP-1 receptor agonist is exenatide. The pharmaceutical formulation may be any described herein or in WO 2010/028257, the disclosure of which is incorporated herein by reference. In one embodiment, the method is for treating overweight. In one embodiment, the method is for treating obesity. In one embodiment, the method is for reducing body weight.

The disclosure provides methods for treating overweight; treating obesity; reducing body weight; treating cardiovascular diseases; treating fatty liver diseases; (e.g., nonalcoholic fatty liver disease (NAFLD); nonalcoholic steatohepatitis (NASH)); treating gastrointestinal diseases; or treating neurodegenerative diseases in patients (e.g., humans) by administering to the patients pharmaceutical formulations comprising (i) a pharmaceutically acceptable non-aqueous carrier; and (ii) microspheres which comprise a biocompatible, biodegradable polymer and a GLP-1 receptor agonist; wherein the GLP-1 receptor agonist is present in an amount of 5 mg to 6 mg; to treat overweight, treat obesity, reduce body weight, treat a cardiovascular disease, treat fatty liver disease (e.g., nonalcoholic fatty liver disease (NAFLD); nonalcoholic steatohepatitis (NASH)); treat a gastrointestinal disease, or treat a neurodegenerative disease in the patients (e.g., humans). In one embodiment, the pharmaceutical formulation is administered to the patient once per month or once every four weeks. In one embodiment where the formulation comprises from 5 mg to 6 mg of the GLP-1 receptor agonist, the once monthly administration of the formulation achieves a therapeutically effective mean steady state plasma concentration GLP-1 receptor agonist of 90 pg/ml to 160 pg/ml for at least one month. In one embodiment where the formulation comprises from 5 mg to 6 mg GLP-1 receptor agonist, the once monthly administration of the formulation achieves a therapeutically effective mean steady state plasma concentration GLP-1 receptor agonist of 100 pg/ml to 150 pg/ml for at least one month. In one embodiment where the formulation comprises from 5 mg to 6 mg GLP-1 receptor agonist, the once monthly administration of the formulation achieves a therapeutically effective mean steady state plasma concentration GLP-1 receptor agonist of 105 pg/ml to 145 pg/ml for at least one month. In one embodiment where the formulation comprises from 5 mg to 6 mg GLP-1 receptor agonist, the once monthly administration of the formulation achieves a therapeutically effective mean steady state plasma concentration GLP-1 receptor agonist of 110 pg/ml to 140 pg/ml for at least one month. In one embodiment where the formulation comprises from 5 mg to 6 mg GLP-1 receptor agonist, the once monthly administration of the formulation achieves a therapeutically effective mean steady state plasma concentration GLP-1 receptor agonist of 115 pg/ml to 135 pg/ml for at least one month. The mean steady state plasma concentration is generally measured at least after administration of 2 doses, preferably after administration of 3 doses of the pharmaceutical formulation. The GLP-1 receptor agonist can be any known in the art, including those described herein. In one embodiment, the GLP-1 receptor agonist is exenatide. The pharmaceutical formulation may be any described herein or in WO 2010/028257, the disclosure of which is incorporated herein by reference. In one embodiment, the method is for treating overweight. In one embodiment, the method is for treating obesity. In one embodiment, the method is for reducing body weight.

The disclosure provides sustained release compositions provided in pharmaceutically acceptable carriers, for the sustained release of an active pharmaceutical ingredient (API). The formulations may comprise microspheres comprised of a biocompatible, biodegradable polymer having an active pharmaceutical ingredient dispersed therein, where the microspheres are suspended in a non-aqueous carrier. The formulations are one-component injectable formulations, compared to two-component formulations which require that the microspheres be stored dry in one container while the liquid carrier can be stored in a separate container, such that the patient must mix the two together prior to injection. The formulations offer the convenience of long term stability of a pharmaceutical composition in a non-aqueous liquid carrier, thus eliminating any need for the patient to add a pharmaceutically acceptable carrier to the pharmaceutical composition prior to injection. The formulations are provided in a single container for easy use by the patient, whom only need to lightly agitate the formulation before injecting it from the same container. When the container provided is also an injection device, even the step of syringing the formulation is eliminated. The formulations described herein offer the additional important advantage of substantially reducing burst release of the active pharmaceutical ingredient. Thus, even active pharmaceutical ingredients that have a toxic effect at higher concentrations can be safely administered using the formulations described herein.

The term “patient” refers to mammals, including humans, animal pets, farm animals, zoo animals, and the like. In one embodiment, the patient is a human.

The terms “treating” or “treatment” refer to the administration of one or more active pharmaceutical ingredients to a patient who has a condition or disorder or a predisposition toward a condition or disorder, with the purpose to alleviate, relieve, remedy, ameliorate, improve, slow or stop the progression or worsening of the disease, or at least one symptom of the disease, condition or disorder, or the predisposition toward the condition or disorder.

The term “cardiovascular diseases” include, for example, myocardial infarction, congestive heart failure, hypertension, hypercholesterolemia, hypertriglyceridemia, ischemic stroke, atherosclerosis, cardiomyopathy, and the like.

The term “gastrointestinal diseases” include, for example, short bowel syndrome.

The term “neurodegenerative diseases” include, for example, Parkinson's disease, Alzheimer's disease, Huntington's disease, and the like.

“Exenatide” has the same meaning and amino acid sequence as exendin-4.

### One Component Formulation

Previous injectable formulations contained at least two components. The first component may be dry microspheres and the second component may be an aqueous pharmaceutically acceptable carrier. The first component and second component are stored in separate sealed containers (e.g., vials, injection pen chambers). The patient receives the two-component formulation, and the patient or pharmacist must physically mix the two components together prior to injection. In the case of an injection pen, the two components are mixed together immediately prior to injection into the patient. Two-component formulations typically are administered to the patient within a short time after being mixed with the pharmaceutically acceptable carrier. For example, the microsphere component and the pharmaceutically acceptable aqueous carrier are mixed together and then the formulation is administered to the patient within about 30 or 60 minutes.

The formulations described herein are one component injectable formulations. A one component injectable formulation refers to a formulation that contains both the microspheres and the pharmaceutically acceptable carrier provided in the same container, and that may be administered to the patient without the need to first combine the microspheres and the pharmaceutically acceptable carrier. Accordingly, the one component formulation is manufactured as a pre-mixed formulation for injection. A one-component formulation provides significant convenience for manufacturing, transport, storage, and patient use.

In another embodiment the one-component formulation described herein is provided in a sealed container. A “sealed container” is a container that has not been opened, punctured, or had anything introduced into it since its time of completion of manufacture. The time of completion of manufacture is the time when the container holding the formulation is initially sealed. Containers may include vials (single use or multi-use), syringes, injection pens (e.g., single use or multi-use), and the like.

### Carrier

“Carrier” (or vehicle) refers to a pharmaceutically acceptable non-aqueous liquid material. The carrier is substantially inert so that it does not interact with the microspheres described herein and is non-toxic so that it does not negatively impact the patient. The carrier is preferably approved by or is awaiting approval by a regulatory agency of the Federal or a state government or listed in the U.S. Pharmacopoeia or other generally recognized pharmacopoeia for use in mammals, such as humans. The term “carrier” may include one or more compounds. The carrier is a non-solubilizing carrier, in that the carrier does not solubilize the polymer(s) that forms the microspheres. In a further embodiment, the carrier does not solubilize the active pharmaceutical ingredient(s) within the microspheres. For example, the carrier will not solubilize exenatide or other water-soluble therapeutic peptides or proteins.

The term “non-aqueous” does not exclude trace amounts of residual water that do not have a demonstrated negative impact on the stability of the sustained release compositions. Thus, a composition may have about 0.1% (w/v) water or even about 0.25% water or less than 0.1% (w/v) water or less than 0.25% (w/v) water and still be considered non-aqueous. The carrier does not solubilize the microspheres to the extent of having a demonstrated negative impact on the stability of the microspheres or demonstrated loss of burst release control. In one embodiment, the carrier does not enter or permeate the biocompatible, biodegradable polymer and is not dispersed within the biocompatible, biodegradable polymer. The carrier also does not cause swelling of the microspheres to an extent that has a demonstrated negative impact on the stability of the microspheres. For example swelling may occur to a degree of less than 1% and still be considered a non-aqueous carrier that is non-swelling of the microspheres.

In one embodiment, the non-aqueous carrier is a pharmaceutically acceptable oil. An oil is a substance that is in a viscous liquid state at ambient temperatures or slightly warmer, and is both hydrophobic (immiscible with water) and lipophilic (miscible with other oils, literally). Exemplary pharmaceutically acceptable oil carriers include vegetable oils and volatile essential oils. Exemplary pharmaceutically acceptable oil carriers include coconut oil, palm oil, palm kernel oil, sesame oil, soybean oil, almond oil, rapeseed oil, corn oil, sunflower oil, peanut oil, olive oil, castor oil, soybean oil, safflower oil, cottonseed oil, ethyl oleate, and the like. The carrier may comprise one oil or a combination of two or more oils.

In one embodiment, the carrier is a fractionated oil or a combination of two or more fractionated oils. Exemplary pharmaceutically acceptable oil carriers include fractionated coconut oil, fractionated palm oil, fractionated palm kernel oil, fractionated sesame oil, fractionated soybean oil, fractionated almond oil, fractionated rapeseed oil, fractionated corn oil, fractionated sunflower oil, fractionated peanut oil, fractionated olive oil, fractionated castor oil, fractionated soybean oil, fractionated safflower oil, fractionated cottonseed oil, and the like. In one embodiment, the carrier is fractionated coconut oil. In one embodiment, the carrier is fractionated palm kernel oil. In one embodiment, the carrier is a combination of fractionated coconut oil and fractionated palm kernel oil.

As used herein, fractionation is a process whereby long chain fatty acids are removed from the oil, such that the resulting fractionated oil substantially comprises medium chain triglycerides. The skilled artisan will appreciate that some long-chain fatty acids may remain in the fractionated oil, but generally in amounts less than 5 wt % or less than 2 wt % of the total fatty acid content of the fractionated oil.

In one embodiment, the carrier is a long chain triglyceride, a medium chain triglyceride, a diglyceride, a monoglyceride, a propylene glycol fatty acid diester, or a combination of two or more thereof.

In one embodiment, the carrier is a medium chain triglyceride. The medium chain triglyceride may be synthetic or natural (e.g., produced from fractionated oils, such as coconut oil and/or palm kernel oil). “Medium chain triglyceride” refers to esters of glycerol having three C₆ to C₁₂ fatty acid chains, where the three fatty acid chains may be the same or different. Medium chain triglycerides are represented by the compound of Formula (I):

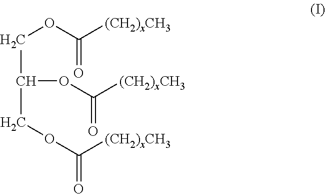

wherein each x is independently 4, 6, 8, or 10. When x is 4, the chain is referred to as a C₆ fatty acid. When x is 6, the chain is referred to as a C₈ fatty acid. When x is 8, the chain is referred to as a C₁₀ fatty acid. When x is 10, the chain is referred to as a C₁₂ fatty acid. In various embodiments, each x is the same integer; two x are the same integer and one x is a different integer; or each x is a different integer.

In various embodiment, the medium chain triglyceride comprises esters of (i) three C₈ fatty acids; (ii) three C₁₋₁₀ fatty acids; (iii) two C₈ fatty acids and one C₁₀ fatty acid; (iv) two C₁₀ fatty acids and one C₈ fatty acid; (v) two C₈ fatty acids and one C₆ fatty acid; (vi) two C₁₀ fatty acids and one C₆ fatty acid; (vii) one C₈ fatty acid, one C₁₀ fatty acid, and one C₆ fatty acid; or (viii) any other combination of C₆, C₈, C₁₀, and C₁₂ fatty acids. In one embodiment, the medium chain triglyceride comprises two C₈ fatty acids and one C₁₀ fatty acid. In one embodiment, the medium chain triglyceride comprises two C₁₀ fatty acids and one C₈ fatty acid.

The skilled artisan will appreciate that a mixture of medium chain triglycerides may result from any process (e.g., fractionation, hydrogenation) used to prepare medium chain triglycerides. For example, substantially all of the medium chain triglycerides obtained from fractionated coconut oil may comprise C₈ and/or C₁₀ fatty acids; however, there may be some medium chain triglycerides containing C₆ and/or C₁₂ fatty acids.

In one embodiment, the medium chain triglycerides comprise esters of (i) 0 to 2 wt % C₆ fatty acid, 65 to 80 wt % C₈ fatty acid, 20 to 35 wt % C₁₀ fatty acid, and 0 to 2 wt % C₁₂ fatty acid; (ii) 0 to 2 wt % C₆ fatty acid, 50 to 65 wt % C₈ fatty acid, 30 to 45 wt % C₁₀ fatty acid, and 0 to 2 wt % C₁₂ fatty acid; (iii) 0 to 2 wt % C₆ fatty acid, 45 to 65 wt % C₈ fatty acid, 30 to 45 wt % C₁₀ fatty acid, 0 to 3 wt % C₁₂ fatty acid; and 0 to 5 wt % linoleic acid; or (iv) 0 to 2 wt % C₆ fatty acid, 45 to 55 wt % C₈ fatty acid, 30 to 40 wt % C₁₀ fatty acid, 0 to 3 wt % C₁₂ fatty acid, and 10 to 20 succinic. In one embodiment, the medium chain triglyceride comprises 0 to 2 wt % C₆ fatty acid, 50 to 65 wt % C₈ fatty acid, 30 to 45 wt % C₁₀ fatty acid, and 0 to 2 wt % C₁₂ fatty acid, and which is commercially available as MIGLYOL® 812 (Sasol Germany GmbH, Witten, Germany) The weight % is based of the total fatty acid content of the triglycerides. In one embodiment, the medium chain triglycerides may comprise up to 2% C₁₄ fatty acids.

The carrier may comprise one, two, three, four or more different medium chain triglycerides. In one embodiment, the carrier comprises a medium chain triglyceride comprising esters of two C₈ fatty acids and one C₁₀ fatty acid. In one embodiment, the carrier comprises a medium chain triglyceride comprising esters of one C₈ fatty acid and two C₁₀ fatty acids. In one embodiment, the carrier comprises two different medium chain triglycerides, where a first medium chain triglyceride comprises esters of two C₈ fatty acids and one C₁₀ fatty acid and a second medium chain triglyceride comprises esters of one C₈ fatty acid and two C₁₀ fatty acids. In one embodiment, the carrier comprises a medium chain triglyceride which comprises 0 to 2 wt % C₆ fatty acid, 50 to 65 wt % C₈ fatty acid, 30 to 45 wt % C₁₀ fatty acid, 0 to 2 wt % C₁₂ fatty acid, based on the total fatty acid content of the medium chain triglyceride.

The triglycerides may be prepared by methods known in the art and are commercially available as MIGLYOL® 810, 812, 818, 829 (Sasol Germany GmbH, Witten, Germany) or NEOBEE® 1053, 895, M-5 (Stepan Company, Northfield, Ill.).

In another embodiment the carrier is a propylene glycol diester of saturated vegetable fatty acids with chain lengths of C₈ and C₁₀ (caprylic and capric acid). An example of one such commercially available carrier is MIGLYOL® 840 (Sasol Germany GmbH, Witten, Germany).

The pharmaceutically acceptable, non-aqueous carrier may optionally comprise other pharmaceutically acceptable excipients. Exemplary excipients include sugars (e.g., sucrose, glucose, dextrose, galactose, maltose, trehalose, fructose, maltodextrin); sugar alcohols (e.g., glycol, glycerol, erythritol, threitol, arabitol, ribitol, sorbitol, dulcitol, iditol, isomalt, maltitol, lactitol, mannitol, xylitol); preservatives (e.g., benzoic acid, sorbic acid, meta cresol, sodium benzoate, potassium sorbate, methylparaben, propylparaben, butylparaben, benzalkonium chloride, and the like, generally oil-soluble, with some solubility in the selected carrier); and antioxidants (e.g., sodium metabisulfite, butylated hydroxy anisole, butylated hydroxy toluene, sodium sulfite, tocopherol, thymol, ascorbate, propyl gallate, and the like). In one embodiment, the carrier optionally comprises mannitol, maltodextrin, sorbitol, or a combination of two or more thereof.

The pharmaceutically acceptable carrier may contain a gel-forming agent; however, the gel-forming agent may only be present in an amount that does not cause a gel-depot to form at the site of in vivo administration of the formulation. In one embodiment, the pharmaceutically acceptable carrier does not contain a gel-forming agent. Exemplary gel-forming agents include cellulose derivatives (e.g., hydroxypropyl cellulose, carboxymethyl cellulose, hydroxyethyl cellulose, hydroxypropyl methyl cellulose, methylcellulose); polyoxyethylene and polyoxypropylene polymers or co-polymers (poloxamers); chitosan acid, and the like. The skilled artisan will understand that the formation of gels in vivo can be determined by methods known in the art, such as the use of histological sections and colored dyes.

In certain embodiments the non-aqueous, non-solubilizing carrier has a viscosity of from 5 cP to 200 cP or from 10 cP to 90 cP. In other embodiments the viscosity of the non-aqueous, non-solubilizing carrier is from 20 cP to 80 cP or from 30 cP to 70 cP. Thus, with reference to this disclosure the person of ordinary skill will be able to identify other oils, triglycerides, or non-aqueous compounds that also can be present in the non-aqueous, non-solubilizing carrier.

### Microspheres

The term “microspheres” includes microspheres, microparticles, nanoparticles, pellets, cylinders, rods, discs, and the like. A microsphere can have a spherical, non-spherical or irregular shape. The microsphere will be of a size suitable for injection. A typical size range for microspheres is 1000 microns or less. In a particular embodiment, the microsphere ranges from about one to about 180 microns in diameter. In yet further embodiments suitable release profiles are obtained when microspheres range from about 1 to 100 microns, from about 30 to 90 microns, or from about 50 to 70 microns. In one embodiment the mean microsphere size is not less than or is equal to about 50, 60 or 70 microns, and preferably less than about 80, 90, or 100 microns. At larger sizes, microsphere are preferably substantially non-aggregated to allow passage through a 25 gauge needle, or a 27 gauge needle, or a 30 gauge needle, or a 31 gauge needle.

Consistent and superior release profiles are obtained by controlling size distribution. In one embodiment a mean microsphere size is about 50 microns and the lower and upper range of microsphere are about 30 and 90 microns, respectively. Distribution of microspheres can be described using a mean diameter of the volume. Mean diameter of the volume distribution represents the center of gravity of the distribution and is a type of “average particle size.” In various embodiments, the microspheres have a mean diameter of the volume distribution of about 50 to 70 microns, about 50 to 60 microns or about 50, 60 or 70 microns, with a Distribution of Volume (DV) of less than or about 5%, 10%, or 15% at 30 microns and a DV of greater than or about 80%, 85%, 90% or 95% at 90 microns. In one embodiment, the microspheres have a mean diameter of the volume distribution of about 60 microns, with a Distribution of Volume (DV) of less than or about 10% at 30 microns and a DV of greater than or about 90% at 90 microns.

Microspheres may be prepared by processes known in the art and described, e.g., in U.S. Pat. Nos. 7,563,871, 7,456,254, 7,223,440, 6,824,822, 6,667,061, 6,495,164, and 6,479,065, the disclosures of which are incorporated by reference herein.

In a further embodiment, the microspheres have a less porous outer layer, and further can have a non-porous outer layer. Accordingly, in the formulations disclosed herein the oil does not have access to the interior spaces or pores or even to a substantial portion of the interior spaces or pores. It is specifically, contemplated that for each of the formulations disclosed herein the microspheres can additionally lack oil (or a carrier as disclosed herein) in the interior spaces of the microspheres. Thus, the advantages of the present formulations can be achieved without the presence of oil in the interior spaces of the microspheres when formulated.

### Polymers

The microspheres comprise biocompatible, biodegradable polymers. A polymer is biocompatible if the polymer and any degradation products of the polymer are non-toxic to the patient at administered levels and also possess no demonstrated deleterious or untoward effects on the patient's body, for example a substantial immunological reaction at the injection site. Biodegradable means the polymer will degrade or erode in vivo to form smaller units or chemical species. Degradation can result, for example, by enzymatic, chemical and physical processes.

Exemplary biocompatible, biodegradable polymers include, for example, polylactides, polyglycolides, poly(lactide-co-glycolides), polylactic acids, polyglycolic acids, poly(lactic acid-co-glycolic acid)s, polycaprolactones, polycarbonates, polyesteramides, polyanhydrides, polyamino acids, polyorthoesters, polycyanoacrylates, poly(p-dioxanone), polyalkylene oxalates, biodegradable polyurethanes, blends thereof and copolymers thereof. Acceptable molecular weights for the biocompatible, biodegradable polymers can be determined by a person of ordinary skill in the art taking into consideration factors such as the desired polymer degradation rate, physical properties such as mechanical strength, end group chemistry and rate of dissolution of polymer. Typically, an acceptable range of molecular weight is of about 2,000 Daltons to about 2,000,000 Daltons. The biocompatible, biodegradable polymer can also be selected based upon the polymer's inherent viscosity. Suitable inherent viscosities are about 0.06 to 1.0 dL/g; about 0.2 to 0.6 dL/g; or about 0.3 to 0.5 dL/g.

In one embodiment, the biocompatible, biodegradable polymer is a poly(lactide-co-glycolide) copolymer (also referred to as “PLGA”) having a lactide:glycolide ratio from 70:30 to 30:70, or from 60:40 to 40:60 or about 50:50. The molecular weight of the poly(lactide-co-glycolide) copolymer is about 10,000 Daltons to about 90,000 Daltons. In another embodiment, the molecular weight of the poly(lactide-co-glycolide) copolymer is about 30,000 Daltons to about 70,000, or from about 50,000 to about 60,000 Daltons.

The formulation may contain microspheres at a concentration of from 1 mg/ml to 500 mg/ml; from 25 mg/ml to 300 mg/ml; or from 50 mg/ml to 200 mg/ml.

### Active Pharmaceutical Ingredient

A “GLP-1 receptor agonist” refers to compounds having GLP-1 receptor activity. Such exemplary compounds include exendins, exendin analogs, exendin agonists, GLP-1(7-37), GLP-1(7-37) analogs, GLP-1(7-37) agonists, and the like. The GLP-1 receptor agonist compounds may optionally be amidated. The terms “GLP-1 receptor agonist” and “GLP-1 receptor agonist compound” are used interchangeably and have the same meaning throughout the specification.

The term “exendin” includes naturally occurring or synthetic versions of naturally occurring exendin peptides that are found in the salivary secretions of the Gila monster. Exendins of particular interest include exendin-3 and exendin-4. The exendins, exendin analogs, and exendin agonists for use in the methods described herein may optionally be amidated, and may also be in an acid form, pharmaceutically acceptable salt form, or any other physiologically active form of the molecule.

Exendin-4 (HGEGTFTSDLSKQMEEEAVRLFIEWLKNGGPSSGAPPPS-NH₂ (SEQ ID NO:1)) is a peptide found in the saliva of the Gila monster, Heloderma suspectum; and exendin-3 (HSDGTFTSDLSKQMEEEAVRLFIEWLKNGG PSSGAPPPS-NH₂ (SEQ ID NO:2)) is a peptide found in the saliva of the beaded lizard, Heloderma horridum. Exendins have some amino acid sequence similarity to some members of the glucagon-like peptide (GLP) family. For example, exendin-4 has about 53% sequence identity with glucagon-like peptide-1(GLP-1) (7-37) (HAEGTFTSDVSSYLEGQAAKEFIAWLVKGRG (SEQ ID NO:22)). However, exendin-4 is transcribed from a distinct gene, not the Gila monster homolog of the mammalian proglucagon gene from which GLP-1 is expressed. Additionally, exendin-4 is not an analog of GLP-1(7-37) because the structure of synthetic exendin-4 peptide was not created by sequential modification of the structure of GLP-1. Nielsen et al, Current Opinion in Investigational Drugs, 4(4):401-405 (2003).

“Exendin analog” refers to peptides which elicit a biological activity of an exendin reference peptide, preferably having a potency equal to or better than the exendin reference peptide (e.g., exendin-4), or within five orders of magnitude (plus or minus) of potency compared to the exendin reference peptide, when evaluated by art-known measures such as receptor binding and/or competition studies as described, e.g., by Hargrove et al, Regulatory Peptides, 141:113-119 (2007), the disclosure of which is incorporated by reference herein. Preferably, the exendin analogs will bind in such assays with an affinity of less than 1 μM, and more preferably with an affinity of less than 3 nM, less than 1 nM, or less than 0.1 nM. In one embodiment, an exendin analog has at least 75% sequence identity to exendin-4. In one embodiment, an exendin analog has at least 80% sequence identity to exendin-4. In one embodiment, an exendin analog has at least 85% sequence identity to exendin-4. In one embodiment, an exendin analog has at least 90% sequence identity to exendin-4. In one embodiment, an exendin analog has at least 95% sequence identity to exendin-4.

Exendin analogs also include the peptides described herein which have been chemically derivatized or altered, for example, peptides with non-natural amino acid residues (e.g., taurine, β-amino acid residues, γ-amino acid residues, and D-amino acid residues), C-terminal functional group modifications, such as amides, esters, and C-terminal ketone modifications and N-terminal functional group modifications, such as acylated amines, Schiff bases, or cyclization, as found, for example, in the amino acid pyroglutamic acid. Exendin analogs may also contain other chemical moieties, such as peptide mimetics.

Exemplary exendins and exendin analogs exendin-4 (SEQ ID NO:1); exendin-3 (SEQ ID NO:2); Leu¹⁴-exendin-4 (SEQ ID NO:3); Leu¹⁴,Phe²⁵-exendin-4 (SEQ ID NO:4); Leu¹⁴,Ala¹⁹,Phe²⁵-exendin-4 (SEQ ID NO:5); exendin-4(1-30) (SEQ ID NO:6); Leu¹⁴-exendin-4(1-30) (SEQ ID NO:7); Leu¹⁴,Phe²⁵-exendin-4(1-30) (SEQ ID NO:8); Leu¹⁴,Ala¹⁹,Phe²⁵-exendin-4(1-30) (SEQ ID NO:9); exendin-4(1-28) (SEQ ID NO:10); Leu¹⁴-exendin-4(1-28) (SEQ ID NO:11); Leu¹⁴,Phe²⁵-exendin-4(1-28) (SEQ ID NO:12); Leu¹⁴,Ala¹⁹,Phe²⁵-exendin-4 (1-28) (SEQ ID NO:13); Leu¹⁴,Lys^{17,20},Ala¹⁹,Glu²¹,Phe²⁵,Gln²⁸-exendin-4 (SEQ ID NO:14); Leu¹⁴,Lys^{17,20},Ala¹⁹,Glu²¹,Gln²⁸-exendin-4 (SEQ ID NO:15); octylGly¹⁴,Gln²⁸-exendin-4 (SEQ ID NO:16); Leu¹⁴,Gln²⁸,octylGly³⁴-exendin-4 (SEQ ID NO:17); Phe⁴,Leu¹⁴,Gln²⁸,Lys³³,Glu³⁴, Ile^{35,36},Ser³⁷-exendin-4(1-37) (SEQ ID NO:18); Phe⁴,Leu¹⁴,Lys^{17,20},Ala¹⁹,Glu²¹,Gln²⁸-exendin-4 (SEQ ID NO:19); Val¹¹,Ile¹³,Leu¹⁴,Ala¹⁶,Lys²¹,Phe²⁵-exendin-4 (SEQ ID NO:20); exendin-4-Lys⁴⁰ (SEQ ID NO:21); lixisenatide (Sanofi-Aventis/Zealand Pharma); CJC-1134 (ConjuChem, Inc.); [N^{e}-(17-carboxyheptadecanoic acid)Lys²⁰]exendin-4-NH₂ (SEQ ID NO: 46); [N^{e}-(17-carboxyhepta-decanoyl)Lys³²]exendin-4-NH₂ (SEQ ID NO: 47); [desamino-His¹,N^{e}-(17-carboxyheptadecanoyl)Lys²⁰]exendin-4-NH₂ (SEQ ID NO: 48); [Arg^{12,27},NLe¹⁴,N^{e}-(17-carboxy-heptadecanoyl)Lys³²]exendin-4-NH₂ (SEQ ID NO: 49); [N^{e}-(19-carboxy-nonadecanoylamino)Lys²⁰]-exendin-4-NH₂ (SEQ ID NO: 50); [N^{e}-(15-carboxypentadecanoylamino)Lys²⁰]-exendin-4-NH₂ (SEQ ID NO: 51); [N^{e}-(13-carboxytridecanoylamino)Lys²⁰]exendin-4-NH₂ (SEQ ID NO: 52); [N^{e}-(11-carboxy-undecanoyl-amino)Lys²⁰]exendin-4-NH₂ (SEQ ID NO: 53); exendin-4-Lys⁴⁰(e-MPA)-NH₂ (SEQ ID NO: 54); exendin-4-Lys⁴⁰(e-AEEA-AEEA-MPA)-NH₂ (SEQ ID NO: 55); exendin-4-Lys⁴⁰(e-AEEA-MPA)-NH₂ (SEQ ID NO: 56); exendin-4-Lys⁴⁰(e-MPA)-albumin (SEQ ID NO: 57); exendin-4-Lys⁴⁰(e-AEEA-AEEA-MPA)-albumin (SEQ ID NO: 58); exendin-4-Lys⁴⁰(e-AEEA-MPA)-albumin (SEQ ID NO: 59); and the like. AEEA refers to [2-(2-amino)ethoxy)]ethoxy acetic acid. EDA refers to ethylenediamine. MPA refers to maleimidopropionic acid. The exendins and exendin analogs may optionally be amidated.

Other exendins and exendin analogs useful in the methods described herein include those described in WO 98/05351; WO 99/07404; WO 99/25727; WO 99/25728; WO 99/40788; WO 00/41546; WO 00/41548; WO 00/73331; WO 01/51078; WO 03/099314; U.S. Pat. No. 6,956,026; U.S. Pat. No. 6,506,724; U.S. Pat. No. 6,703,359; U.S. Pat. No. 6,858,576; U.S. Pat. No. 6,872,700; U.S. Pat. No. 6,902,744; U.S. Pat. No. 7,157,555; U.S. Pat. No. 7,223,725; U.S. Pat. No. 7,220,721; US Publication No. 2003/0036504; and US Publication No. 2006/0094652, the disclosures of which are incorporated by reference herein in their entirety.

“GLP-1(7-37) analogs” refers to peptides which elicit a biological activity similar to that of GLP-1(7-37), when evaluated by art-known measures such as receptor binding assays or in vivo blood glucose assays as described, e.g., by Hargrove et al, Regulatory Peptides, 141:113-119 (2007), the disclosure of which is incorporated by reference herein. In one embodiment, the term “GLP-1(7-37) analog” refers to a peptide that has an amino acid sequence with 1, 2, 3, 4, 5, 6, 7 or 8 amino acid substitutions, insertions, deletions, or a combination of two or more thereof, when compared to the amino acid sequence of GLP-1(7-37). In one embodiment, the GLP-1(7-37) analog is GLP-1(7-36)-NH₂. GLP-1(7-37) analogs include the amidated forms, the acid form, the pharmaceutically acceptable salt form, and any other physiologically active form of the molecule. In one embodiment, a GLP-1 analog has at least 75% sequence identity to GLP-1(7-37). In one embodiment, a GLP-1 analog has at least 80% sequence identity to GLP-1(7-37). In one embodiment, a GLP-1 analog has at least 85% sequence identity to GLP-1(7-37). In one embodiment, a GLP-1 analog has at least 90% sequence identity to GLP-1(7-37). In one embodiment, a GLP-1 analog has at least 95% sequence identity to GLP-1(7-37).

Exemplary GLP-1(7-37) and GLP-1(7-37) analogs include GLP-1(7-37) (SEQ ID NO:22); GLP-1(7-36)-NH₂ (SEQ ID NO:23); liraglutide (VICTOZA® from Novo Nordisk); albiglutide (SYNCRIA® from GlaxoSmithKline); taspoglutide (Hoffman La-Roche); LY2189265 (Eli Lilly and Company); LY2428757 (Eli Lilly and Company); desamino-His⁷,Arg²⁶,Lys³⁴(N^{ε}-(γ-Glu(N-α-hexadecanoyl)))-GLP-1(7-37) (core peptide disclosed as SEQ ID NO: 60); desamino-His⁷,Arg²⁶,Lys³⁴(N^{ε}-octanoyl)-GLP-1(7-37) (SEQ ID NO: 61); Arg^{26,34},Lys³⁸(N^{ε}-(ω-carboxypentadecanoyl))-GLP-1(7-38) (SEQ ID NO: 62); Arg^{26,34},Lys³⁶(N^{ε}-(γ-Glu(N-α-hexadecanoyl)))-GLP-1(7-36) (core peptide disclosed as SEQ ID NO: 63); Aib^{8,35},Arg^{26,34},Phe³¹-GLP-1(7-36)) (SEQ ID NO:24); HXaa₈EGTFTSDVSSYLEXaa₂₂Xaa₂₃AAKEFIXaa₃₀WLXaa₃₃Xaa₃₄G Xaa₃₆Xaa₃₇; wherein Xaa₈ is A, V, or G; Xaa₂₂ is G, K, or E; Xaa₂₃ is Q or K; Xaa₃₀ is A or E; Xaa₃₃ is V or K; Xaa₃₄ is K, N, or R; Xaa₃₆ is R or G; and Xaa₃₇ is G, H, P, or absent (SEQ ID NO:25); Arg³⁴-GLP-1(7-37) (SEQ ID NO:26); Glu³⁰-GLP-1(7-37) (SEQ ID NO:27); Lys²²-GLP-1(7-37) (SEQ ID NO:28); Gly^{8,36},Glu²²-GLP-1(7-37) (SEQ ID NO:29); Val⁸,Glu²²,Gly³⁶-GLP-1(7-37) (SEQ ID NO:30); Gly^{8,36},Glu²²,Lys³³,Asn³⁴-GLP-1(7-37) (SEQ ID NO:31); Val⁸,Glu²²,Lys³³,Asn³⁴,Gly³⁶-GLP-1(7-37) (SEQ ID NO:32); Gly^{8,36},Glu²²,Pro³⁷-GLP-1(7-37) (SEQ ID NO:33); Val⁸,Glu²²,Gly³⁶Pro³⁷-GLP-1(7-37) (SEQ ID NO:34); Gly⁸³⁶,Glu²²,Lys³³, Asn³⁴,Pro³⁷-GLP-1(7-37) (SEQ ID NO:35); Val⁸,Glu²²,Lys³³,Asn³⁴,Gly³⁶,Pro³⁷-GLP-1(7-37) (SEQ ID NO:36); Gly^{8,36},Glu²²-GLP-1(7-36) (SEQ ID NO:37); Val⁸,Glu²²,Gly³⁶-GLP-1(7-36) (SEQ ID NO:38); Val⁸,Glu²²,Asn³⁴,Gly³⁶-GLP-1(7-36) (SEQ ID NO:39); Gly^{8,36},Glu²²,Asn³⁴-GLP-1(7-36) (SEQ ID NO:40). Each of the GLP-1(7-37) and GLP-1(7-37) analogs may optionally be amidated.

In one embodiment, the GLP-1(7-37) or GLP-1(7-37) analogs are covalently linked (directly or by a linking group) to an Fc portion of an immunoglobulin (e.g., IgG, IgE, IgG, and the like). For example, any one of SEQ ID NOs:25-40 may be covalently linked to the Fc portion of an immunoglobulin comprising the sequence of: AESKYGPPCPPCPAPXaa₁₆ Xaa₁₇Xaa₁₈ GGPSVFLFPPKPKDTLMISRTPEVTCVVVDVSQEDPEVQFNWYVDGVEVH NAKTKPREEQFXaa₈₀STYRVVSVLTVLHQDWLNGKEYKCKVSNKGLPSSIEKTISKAKG QPREPQVYTLPPSQEEMTKNQVSLTCLVKGFYPSDIAVEWESNGQPENNYKTTPPVLD SDGSFFLYSRLTVDKSRWQEGNVFSCSVMHEALHNHYTQKSLSLSLGXaa₂₃₀; wherein Xaa₁₆ is P or E; Xaa₁₇ is F, V or A; Xaa₁₈ is L, E or A; Xaa₈₀ is N or A; and Xaa₂₃₀ is K or absent (SEQ ID NO:41). The linking group may be any chemical moiety (e.g., amino acids and/or chemical groups). In one embodiment, the linking group is (-GGGGS-)ₓ (SEQ ID NO:42) where x is 1, 2, 3, 4, 5 or 6; preferably 2, 3 or 4; more preferably 3. In one embodiment, the GLP-1(7-37) analog covalently linked to the Fc portion of an immunoglobulin comprises the amino acid sequence: HGEGTFTSDVSSYLEEQAAKEFIAWLVK GGGGGGGSGGGGSGGGGSAE SKYGPPCPPCPAPEAAGGPSVFLFPPK PKDTLMISRTPEVTCVVVDVSQEDPE VQFNWYVDGVEVHNAKTKPREEQFNSTYR VVSVLTVLHQDWLNGKEYKCK VSNKGLPSSIEKTISKAKGQPREPQVYTL PPSQEEMTKNQVSLTCLVKGFYPSDI AVEWESNGQPENNYKTTPPVLDSDGSFFLYSR LTVDKSRWQEGNVFSCSVMHEA LHNHYTQ KSLSLSLG (SEQ ID NO:43).

In another embodiment, the GLP-1(7-37) or GLP-1(7-37) analog may be covalently linked (directly or through a linking group) to one or two polyethylene glycol molecules. For example, a GLP-1(7-37) analog may comprise the amino acid sequence: HXaa₈EGTFTSDVS SYLEXaa₂₂QAAKEFIAWLXaa₃₃KGGPSSGAPPPC₄₅C₄₆-Z, wherein Xaa₈ is: D-Ala, G, V, L, I, S or T; Xaa₂₂ is G, E, D or K; Xaa₃₃ is: V or I; and Z is OH or NH₂, (SEQ ID NO:44), and, optionally, wherein (i) one polyethylene glycol moiety is covalently attached to C₄₅, (ii) one polyethylene glycol moiety is covalently attached to C₄₆, or (iii) one polyethylene glycol moiety is attached to C₄₅ and one polyethylene glycol moiety is attached to C₄₆. In one embodiment, the GLP-1(7-37) analog is HVEGTFTSDVSSYLEEQAAKEFI AWLIKGGPSSGAPPPC₄₅C₄₆-NH₂ (SEQ ID NO:45) and, optionally, wherein (i) one polyethylene glycol moiety is covalently attached to C₄₅, (ii) one polyethylene glycol moiety is covalently attached to C₄₆, or (iii) one polyethylene glycol moiety is attached to C₄₅ and one polyethylene glycol moiety is attached to C₄₆.

The term amylin agonist refers to a compound which elicits a biological effect similar to that of native amylin, for example a compound (1) having activity in a food intake, gastric emptying, pancreatic secretion, or weight loss assay (PCT Application No. PCT/US2005/004631, filed on Feb. 11, 2005, and incorporated by reference) similar to the native human reference peptide, and/or (2) which binds specifically in a reference receptor assay or in a competitive binding assay with amylin. In one embodiment, the agonists will bind in such assays with an affinity of better than 1 μM, and, in another embodiment, with an affinity of better than 1-5 nM. Exemplary amylin agonists include pramlintide, davalintide, and a peptide having the amino acid sequence KCNTATCVLGRLSQELHRLQTYPRTNVG SNTY-NH₂ (SEQ ID NO: 64)

GLP-1 receptor agonist compounds and amylin agonists may be prepared by processes well known in the art, e.g., peptide purification as described in Eng et al, J. Biol. Chem., 265:20259-62 (1990); standard solid-phase peptide synthesis techniques as described in Raufman et al, J. Biol. Chem., 267:21432-37 (1992); recombinant DNA techniques as described in Sambrook et al, Molecular Cloning: A Laboratory Manual, 2d Ed., Cold Spring Harbor (1989); and the like.

### Sugars

The microspheres may also comprise one or more sugars. A sugar is a monosaccharide, disaccharide or oligosaccharide or a derivative thereof. Sugar alcohols of monosaccharides are suitable derivatives of sugar. Monosaccharides include, but are not limited to, glucose, fructose and mannose. A disaccharide, as further defined herein, is a compound which upon hydrolysis yields two molecules of a monosaccharide. Suitable disaccharides include, but are not limited to, sucrose, lactose and trehalose. Suitable oligosaccharides include, but are not limited to, raffinose and acarbose. The microspheres may further comprise glucose, dextrose, galactose, maltose, fructose, mannose, sucrose, lactose, trehalose, raffinose, acarbose, glycol, glycerol, erythritol, threitol, arabitol, ribitol, sorbitol, dulcitol, iditol, isomalt, maltitol, lactitol, mannitol, xylitol, or a combination of two or more thereof. In one embodiment, the sugar is sucrose, glucose, mannose, or fructose. In one embodiment, the sugar is sucrose.

The amount of sugar present in the microspheres can range from about 0.01% (w/w) to about 50% (w/w), such as from about 0.01% (w/w) to about 10% (w/w), such as from about 0.1% (w/w) to about 5% (w/w) of the total weight of the composition. In one embodiment, about 2% (w/w) sucrose is used.

Alternatively, the amount of sugar present in the microspheres can be referred to on a weight ratio with the active pharmaceutical ingredient. For example, the active pharmaceutical ingredient and sugar can be present in a ratio from about 10:1 to about 1:10 weight:weight. In particularly preferred embodiments, the ratio of active pharmaceutical ingredient (e.g., exenatide) to sugar (e.g., sucrose) is about 3:2 (w/w), 4:2 (w/w), or 5:2 (w/w). Combinations of two or more sugars can also be used. The amount of sugar, when a combination is employed, is the same as the ranges recited above.

### Sustained Release

The compositions are sustained release compositions, meaning that the active pharmaceutical ingredient contained in the compositions will be released into the patient over an extended period of time such as, for example, two weeks, three weeks, one month, three months, or one year. The release of the active pharmaceutical ingredient is considered complete when there is no longer a therapeutic level of active pharmaceutical ingredient in the patient's body, as determined by the medical judgment of those of ordinary skill in the art.

Cₘₐₓ as used herein is the maximum serum concentration of drug which occurs during the period of release which is monitored. Cₐᵥₑ as used herein, is the average serum concentration of drug derived by dividing the area under the curve (AUC) of the release profile by the duration of the release.

In one embodiment the ratio of Cₘₐₓ to Cₐᵥₑ is about 3 or less. This profile is particularly desirable for anti-diabetic or glucoregulatory polypeptides, such as those described herein. A ratio of about 3 or less can provide a Cₐᵥₑ in a therapeutic window while avoiding adverse drug side effects which can result from higher ratios. Further by controlling the physical aspects of the sustained release composition, as described herein, a superior desired release profile can be achieved and controlled, for example, by appropriate selection of carrier properties, such as viscosity. There is thus provided a reduced burst (i.e. initial release; e.g., Cₘₐₓ at 0-1 day). In other embodiments the Cₘₐₓ to Cₐᵥₑ ratio is from about 1 to about 3, or from 1 to 3, or from about 2 to about 3, or from 2 to 3. Further, a Cₘₐₓ, if present, can be shifted from the burst or initial release period into the “sustained phase” of release. In one embodiment the Cₘₐₓ can occur at at least 7, 14, 21, 28, 35 or 42 days post administration and can occur at any integer day in between. In a further embodiment the Cₘₐₓ occurs at about 21 to 35 days after administration, and in yet another embodiment is at about 28 to 31 days, and further at about 28 days after administration. In a further embodiment the maximal concentration of drug (e.g. plasma concentration) occurs at least 7, 14, 21, 28, 35 or 42 days post administration and can occur at any integer day in between. In yet a further embodiment the maximal concentration of drug occurs at about 21 to 35 days after administration.

### Longer Shelf Life

One advantage offered by the present formulations is a longer shelf life for the formulation. It was discovered unexpectedly that sustained release compositions retain remarkable stability when stored in a non-aqueous carrier as described herein. In one embodiment the formulation has a shelf life of at least 6 months. In other embodiments the formulation has a shelf life of at least 1 year, or at least 18 months, or at least 2 years. By “shelf life” is meant the formulation can be stored or maintained for that period of time under appropriate environmental conditions while retaining at least 90% of the desired activity of the active pharmaceutical ingredient relative to the activity at initial formulation (as 100%). In another embodiment the active pharmaceutical ingredient retains at least 95%, or at least 98% or at least 99% of its desired activity as compared to its activity immediately before storage. When the formulation contains microspheres, shelf life also refers to the retention of particle size and/or morphology of the microspheres. Retention of size morphology can be determined by microscopic examination, the use of which is known to persons of ordinary skill in the art.

When formulated as disclosed herein a peptide or protein as active ingredient is less susceptible to oxidation and to hydrolysis, either chemical or proteolytic, both during storage and during its sustained release period after injection. The addition of an anti-oxidant or other stabilizer is not required in these formulations, particularly those where the carrier is a medium chain triglyceride.

### Reduced Burst Release

Another advantage of the present formulations is that formulations according to the present disclosure offer a significantly reduced burst release rate compared with other formulations. When previously available injectable sustained release formulations are injected into a patient there is often a “burst” of active ingredient or agent associated with the injection. Without wanting to be bound by any specific theory, it is believed this burst is caused by that quantity of active pharmaceutical ingredient in the formulation that is not retained within the polymer that is released over time. By “burst release” is meant that quantity of active pharmaceutical ingredient released within the first 24 hours after injection. In other embodiments it is that quantity of active that is release over 1 hour, or 2 hours, or 4 hours, or 8 hours, or 12 hours after injection. In various embodiments the formulation of the invention has a burst release after injection of less than 10% or less than 5%, or less than 3%, or less than 2.5%, or less than 2%, or less than 1% or less than 0.75% or less than 0.5% or less than 0.25% or less than 0.1%. Percentages refer to the percentage of the total amount of active pharmaceutical ingredient in the injected formulation. Following injection of the formulation in the patient, the burst release may occur at any time up to about 24 hours, thereafter there may be a lag time where substantially no active pharmaceutical ingredient is released from the microspheres, and then the polymeric microspheres begin degrading and releasing the active pharmaceutical ingredient. The skilled artisan will appreciate that the time period when the burst release occurs may vary from patient to patient.

Burst can be assessed by measuring the proportion of the total area under the curve for a particular time period following administration of a drug. Area under the curve (AUC) is a well established measurement in the pharmaceutical sciences and measures the amount of drug or active ingredient that reaches the bloodstream in a set period of time. As is well known in the art, the period of time selected will varying depending on the time period the concentration of the drug in the blood is expected to be detectable or within the drug's therapeutic window. AUC is calculated by plotting the concentration of the drug in the blood, for example plasma concentrations, at various times during the selected time period and then calculating the total area under the curve obtained. In one exemplary embodiment, the area under the curve is measured for a 42 day period and using the formulations described herein, the release or burst as measured within the first 24 hours is 5% or less, 2% or less, 1.5% or less, 1% or less, or 0.5% or less of the total AUC. In another embodiment, the formulations described herein result in a burst or proportion of the AUC that is 20% or less, 15% or less, 10% or less, 5% or less, or 2% or less than that obtained when the sustained release composition is contained in a carrier in which the active pharmaceutical ingredient is soluble.

In another embodiment, the formulations described herein limit initial burst such that the upper limit of the therapeutic window for the active pharmaceutical ingredient is not exceeded. The therapeutic window is the range of concentration of active pharmaceutical ingredient in the circulation, above which the active pharmaceutical ingredient has its desired effect, but below the concentration at which the adverse effects associated with the active pharmaceutical ingredient outweigh the benefits as would be generally accepted among physicians. In one exemplary embodiment, the active pharmaceutical ingredient is an exendin, for example exenatide, or agonist analogue thereof, and administration of the formulations described do not result in a circulating level of active pharmaceutical ingredient exceeding 400 pg/ml during the first 24 hours following administration. In another exemplary embodiment the active pharmaceutical ingredient is an exendin, for example exenatide, or agonist analogue thereof, and administration of the formulations described does not result in a circulating level of active pharmaceutical ingredient exceeding 350 pg/ml during the first 24 hours following administration.

Initial burst can also be assessed by comparing circulating concentrations of the active pharmaceutical ingredient in a time period immediately following administration of the formulation with the circulating concentration of the drug in a second time period that immediately follows the first. In one embodiment, use of the formulations of the present disclosure result in circulating concentrations of active pharmaceutical ingredient during the first 24 hours following administration that do not exceed the circulating concentration during the next 24 hour period. In another embodiment, use of the formulations of the present disclosure result in average circulating concentration of active pharmaceutical ingredient during the first 24 hours following administration do not exceed the average circulating concentration during the next 24 hour period.

### Methods of Storing

Another aspect provides methods of storing the sustained release formulations described herein. The methods of storing the formulations described herein may also be referred to as methods of preventing the degradation of the microspheres. By “storing” is meant that the formulation is retained for a period of time within its container without adding any additional component to the container and without removing the formulation from the container (e.g., in the manufacturing facility, during transport, in the pharmacy). The storage time will typically be the amount of time between packaging of the formulation and its use by the patient. After the storage time the formulation is administered to the patient in need thereof. “Administering” to the patient includes self-administration. The methods involve storing the sustained release formulations for a period of at least 1 week, at least 2 weeks, at least 1 month, at least 3 months, at least 1 year, at least 18 months, or at least 2 years. In some embodiments, the formulations can be stored at 5° C. or 25° C. There is minimal degradation of the microspheres when the formulations are stored for such extended periods of time.

In another embodiment the invention provides methods of maintaining the potency of (e.g., preventing the loss of biological activity) and/or purity (e.g. preventing chemical changes in the molecule) an active pharmaceutical ingredient. Thus, a peptide or protein or other API that has undergone a chemical change (e.g. oxidation) may result in a loss of purity, but may still retain its potency. The methods involve storing a microsphere comprising a active pharmaceutical ingredient in a non-aqueous carrier as described herein for a period of time, whereby the potency and/or purity of the active pharmaceutical ingredient is maintained by the microspheres and the non-aqueous carrier. In the formulations described herein, at least 80%, at least 90%; at least 95%; at least 98%; or at least 99% of the potency and/or purity of the active pharmaceutical ingredient is retained for a period of time of at least 1 week, at least 2 weeks, at least 1 month, at least 3 months, at least 1 year, at least 18 months, or at least 2 years.

### Methods of Administering/Treatment

In another aspect the present invention provides methods of administering an active pharmaceutical ingredient to a patient in need thereof. The methods involve administering to the patient a formulation or composition as described herein. Any of the formulations described herein can be administered by parenteral administration, using any of the methods described herein. For example, the formulations can be administered by subcutaneous, intra-muscular, intra-peritoneal, intra-abdominal, intravenous, or any suitable manner of administration. In one embodiment, the formulations described herein are administered subcutaneously. In one embodiment the methods involve injecting the formulation without the patient performing a prior step of combining the sustained release composition with a second carrier.

In one embodiment the administration does not comprise a mixing step. A mixing step is a step where the microspheres are combined with a carrier prior to injection. In various embodiments the mixing step is a step where the microspheres are combined with a carrier within the 1 week period prior to injection in the patient. The carrier can be a non-aqueous carrier, such as those described herein. Administration of the formulation refers to the complete process of the user interacting with the formulation, including mixing, combining any ingredients forming the formulation, and the actual injection or other form of providing the formulation to the patient.

The frequency of administration can vary depending on any one or a combination of factors such as the amount of the formulation administered, the release profile of the formulation, the amount of active pharmaceutical ingredient in the formulation, and the circulating level of active pharmaceutical ingredient to be achieved. In particular embodiments, the formulations described herein can be administered once daily, once per week, once every two weeks, once a month, once every two months, once every three months, once every four months, once every six months or once per year. In one embodiment, the formulation is administered once a week. In another embodiment, the formulation is administered once a month.

The formulations described herein can be used to treat numerous diseases, such as diabetes (e.g., Type 1 diabetes, Type II diabetes, gestational diabetes), impaired glucose tolerance, hyperglycemia (e.g., fasting and postprandial), obesity, overweight, fatty liver disease (e.g., non-alcoholic fatty liver disease (NAFLD), non-alcoholic steatohepatitis (NASH)), cardiovascular diseases and the like. The formulations described herein will also be useful to stimulate insulin release; lower plasma glucagon; reduce food intake, reduce appetite, decrease gastric motility, delay gastric emptying, lower plasma lipid (e.g., triglycerides, cholesterol) levels, and the like. These methods of treatment are described, for example, in U.S. Pat. No. 5,424,286, U.S. Pat. No. 6,858,576, U.S. Pat. No. 6,872,700, U.S. Pat. No. 6,956,025, and U.S. Pat. No. 6,956,025, and WO 2007/022518, the disclosures of which are incorporated by reference herein.

The formulations and methods described herein are particularly useful in delaying gastric emptying, and in the treatment of diseases and disorders that benefit by the delay of gastric emptying, e.g., the treatment of diabetes. In one embodiment, the unique release profile (e.g., sawtooth profile) achieved by dosing once per month (e.g., once every four weeks) contributes to delaying gastric emptying and to treating diabetes. The delay in gastric emptying provided can be more than that provided by either EQW or EQWS that achieve an essentially smooth PK profile absent repeating peaks and troughs. The delay in gastric emptying will provide a noticeable reduction in post-prandial glucose plasma levels.

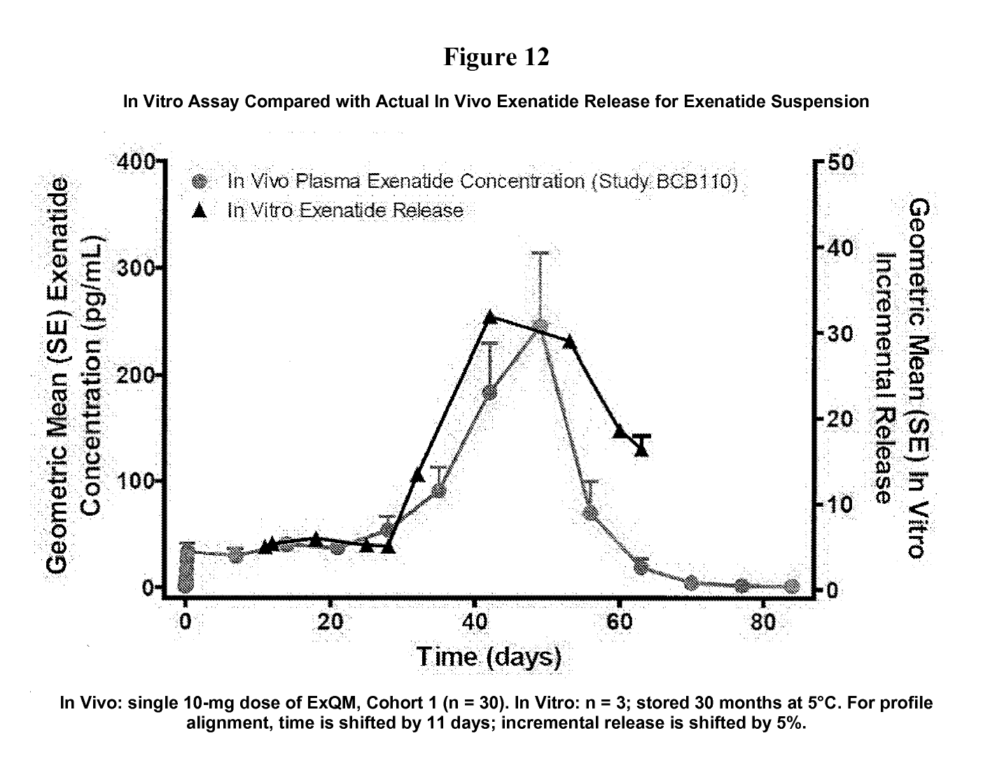

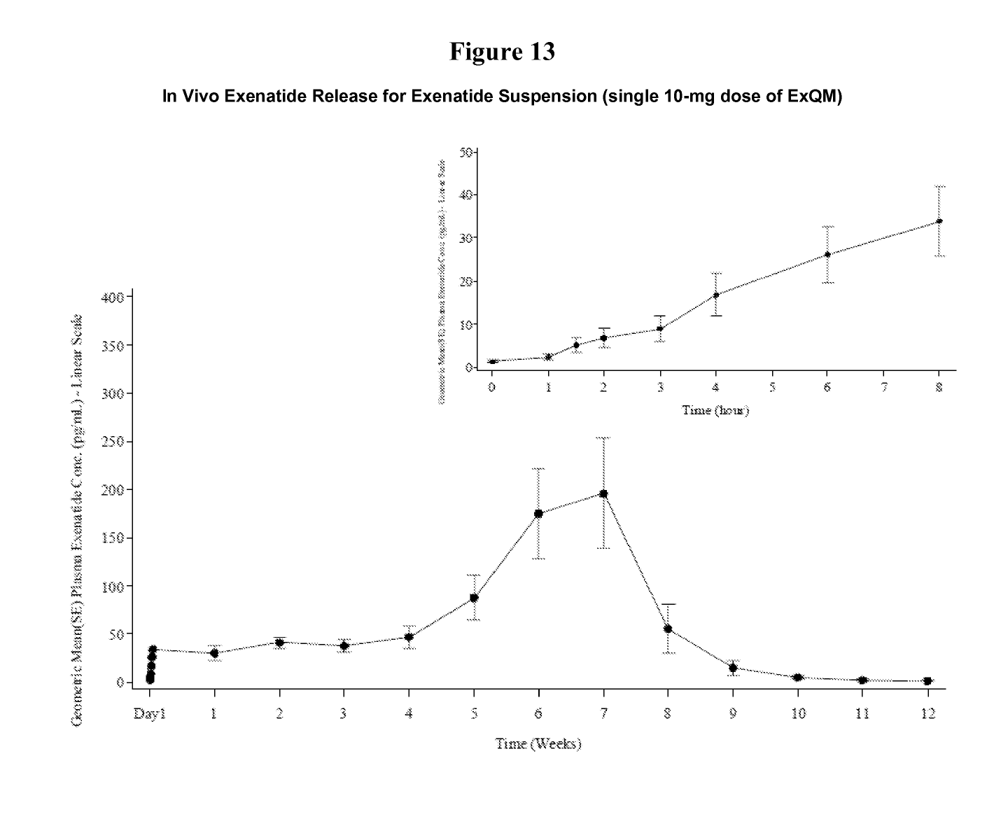

The human in vivo release profile for a single dose of the formulations described herein show a small transient rise over the first 8 hours, followed by a plateau, and one large peak at about 6-7 weeks, wherein about 70% of exenatide or the GLP-1 receptor agonist is released between weeks 4 and 8, the Tₘₐₓ occurs in the large peak at about 42-49 days, and less than 0.5% of the exenatide or the GLP-1 receptor agonist is released within the first 24 hours after injection. See FIGS. 12-13. The Cₘₐₓ, depending on the dosage amount, is at least 60 pg/ml, 75 pg/ml, 100 pg/ml, 125 pg/ml, 150 pg/ml, 175 pg/ml, 200 pg/ml, 225 pg/ml, 225 pg/ml, 250 pg/ml, 275 pg/ml, or 300 pg/ml.

The human in vivo release profile for the monthly dosing of the formulations described herein show a maximum plasma concentrations at approximately 2 weeks after each monthly administration. The maximum plasma concentrations can range from 150 pg/mL-500 pg/mL, 200 pg/mL-500 pg/mL, 250 pg/mL-500 pg/mL, or 255 pg/mL-500 pg/mL. Within the dosing interval, plasma concentration may decline to a minimum level of approximately 50 pg/, depending on the dosage amount. The peak to trough ratio of exenatide at steady state range from 5-9, 5-8, or about 5, 6, 7, 8, or 9.

As mentioned above, the methods described herein are useful in the treatment of many diseases or disorders. Without being bound by any particular theory, one aspect believed to contribute to the treatment of the diseases and disorders is the unique release profile associated with monthly dosing. The monthly dosing results in a sawtooth-type release profile, wherein the peak to trough ratios are as described herein, e.g., from 5-9, 5-8, or about 5, 6, 7, 8, or 9. In one embodiment, the unique release profile associated with monthly dosing contributes to the delay in gastric emptying, and in the treatment of diseases and disorders that benefit by the delay of gastric emptying, e.g., the treatment of diabetes.

### EXAMPLES

The following non-limiting examples provide further illustrations of making and using the formulations described herein, and are not intended to limit the scope of the appended claims. With respect to the Examples herein, MCT oil refers to medium chain triglyceride oil which is commercially available as MIGLYOL® 812 (Sasol Germany GmbH, Witten, Germany).

### Example 1

Microspheres may be prepared by processes known in the art and described, e.g., in U.S. Pat. No. 7,563,871 and U.S. Pat. No. 7,456,254. Microspheres comprising a poly(lactide-co-glycolide) copolymer having dispersed therein 5% (w/w) exenatide and 2% (w/w) sucrose were obtained. The poly(lactide-co-glycolide) copolymer had a ratio of lactide:glycolide of 1:1. These microspheres are currently being developed by Amylin Pharmaceuticals, Inc. (San Diego, Calif.), Alkermes, Inc. (Cambridge, Mass.), and Eli Lilly and Company (Indianapolis, Ind.) for a once-weekly formulation for treating diabetes. Gedulin et al, Diabetologia, 48:1380-1385 (2004).

### Example 2

The stability of the microspheres from Example 1 was investigated to determine their stability over an extended period of time while stored in a non-aqueous carrier. Microspheres from Example 1 were stored for a period of 6 months at 5° C. in a formulation comprising a non-aqueous carrier (i.e., sesame oil; MCT oil; and ethyl oleate, which is a monoglyceride). The control was an aqueous formulation comprising the microspheres from Example 1 in an aqueous carrier containing carboxymethylcellulose and a surfactant.

The stability of the microspheres was determined by morphology and particle size via examination under a microscope. Exenatide purity, potency (by HPLC evaluation), and in vitro release were also determined. As shown in Table 1, after 6 months of storage the physical structure (i.e., size, morphology) of the microspheres did not change.

As shown in Table 2, the microspheres stored in a MCT oil showed no change in the purity of exenatide based on an HPLC analysis. Impurities might also be referred to as degradation products from the peptide. High purity means relatively little degradation of the peptide. The purity is relative to the formulation at time zero. The microspheres stored in sesame oil and ethyl oleate showed a slight decrease in the purity of exenatide. The impurities did not appear to be oil or poly(lactide-co-glycolide) polymer related (based on retention times), but appeared to be related to the stability of exenatide itself

Table 3 shows that the potency of exenatide did not significantly decrease over the 6 month period regardless of the non-aqueous carrier that was used.

TABLE 1
Particle size and morphology using microscope

| | size (μm) | | | |
|---|---|---|---|---|
| | (standard deviation (μm)) | | | morphology |
| | T = 0 | 1 month | 6 months | 0 to 6 months |
| sesame | 64 (22) | 63 (23) | 64 (12) | no change |
| oil | | | | |
| MCT oil | 65 (19) | 60 (22) | 61 (17) | no change |
| ethyl | 64 (16) | 62 (16) | 59 (13) | no change |
| oleate | | | | |

TABLE 2
Change in Purity of Exenatide Containing Formulation

| | % purity of exenatide | | | | | | |
|---|---|---|---|---|---|---|---|
| | | 1 | % | 3 | % | 6 | % |
| | t = 0 | month | change* | month | change* | month | change* |
| sesame | 95.93 | 95.68 | −0.25 | 94.55 | −1.38 | 95.00 | −0.93 |
| oil | | | | | | | |
| MCT | 95.63 | 95.56 | −0.07 | 94.67 | −0.96 | 95.50 | −0.13 |
| oil | | | | | | | |
| ethyl | 95.60 | 95.80 | 0.20 | 93.67 | −1.93 | 94.70 | −0.90 |
| oleate | | | | | | | |

*Changes less than 0.5% are considered to be insignificant

TABLE 3
Change in Potency of Exenatide Based on Carrier in Formulation

| | time | | | |
|---|---|---|---|---|
| carrier | zero | 1 month | 3 months | 6 months |
| sesame oil | 97 | 104 | 98 | 98 |
| MCT oil | 94 | 108 | 99 | 99 |
| ethyl oleate | 95 | 98 | 99 | 100 |

### Example 3

The pharmacokinetics of the formulations in Example 2 were determined, except that 2% (w/w) lecithin was added to the ethyl oleate carrier. Single injections with a dose of 53 mg/ml of microspheres per ml of non-aqueous carrier were administered to 6 rats with a 21 G needle. In the study, a comparison was also made to the microspheres from Example 1 that were mixed with an aqueous carrier just before injection.

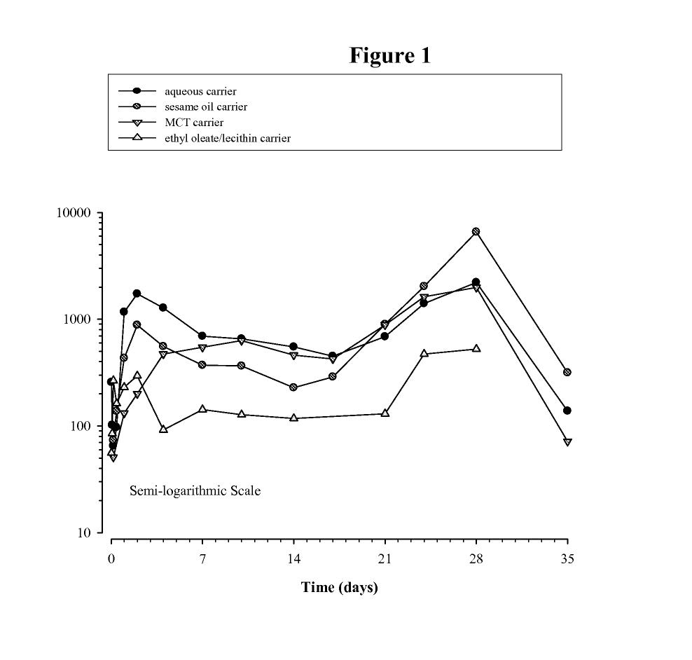

FIG. 1 provides a comparison of the pharmacokinetics of the four different formulations of microspheres containing exenatide. In three formulations, the carrier is an oil (e.g., sesame oil; MCT oil; ethyl oleate). In one comparative formulation, the carrier is an aqueous diluent. As can be seen from the data, the formulations having an oil carrier had reduced burst when compared to the formulation having an aqueous carrier.

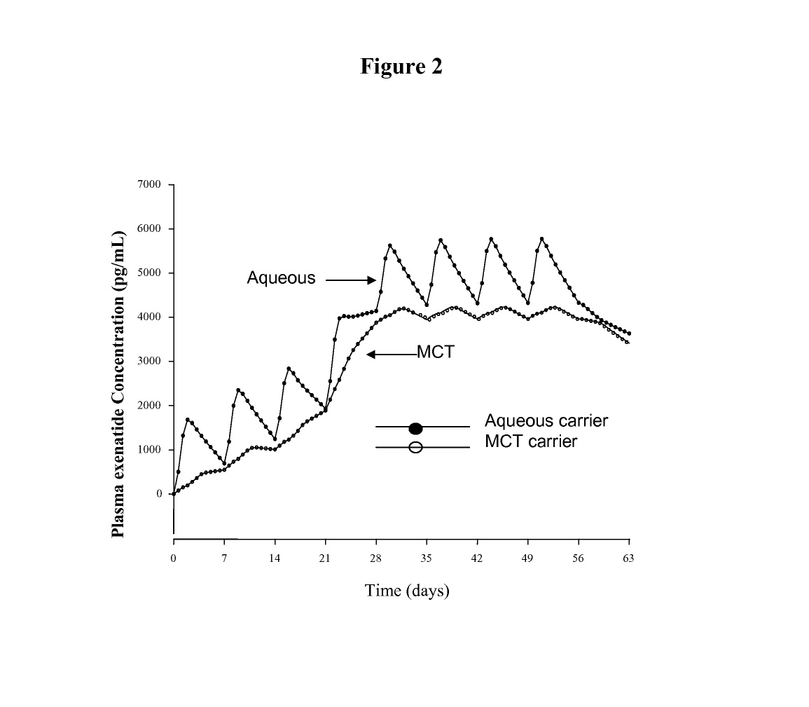

FIG. 2 is a graphical simulation of data extrapolated from FIG. 1 of the plasma exenatide concentration over time of the formulation comprising the MCT oil carrier and the comparative formulation comprising the aqueous carrier. The plasma concentration plateau of exenatide may be reached after about 5 dosings.

### Example 4

A formulation comprising the microspheres of Example 1 in an aqueous carrier and a formulation comprising the microspheres of Example 1 in an MCT carrier were prepared. The burst release was evaluated by adding about 0.75 mL of the formulations to a 10 mM HEPES release buffer. The mixture was agitated to ensure that the microspheres achieved full contact with the HEPES release buffer. After incubation at 37° C. for one hour, the mixture was centrifuged and the aqueous phase was analyzed by HPLC to determine the burst release. The concentration of the dose tested for release was 150 mg/mL.

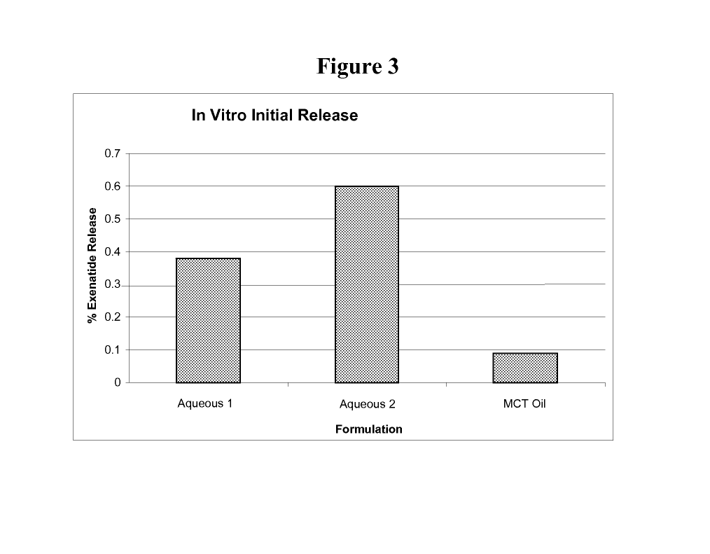

FIG. 3 shows the lower burst release of the formulation having the oil carrier compared to the formulations having an aqueous carrier. The graph shows that with an aqueous carrier, about 0.6% of exenatide was released in the burst. With the formulation having the MCT oil carrier, less than 0.1% of exenatide was released in the burst.

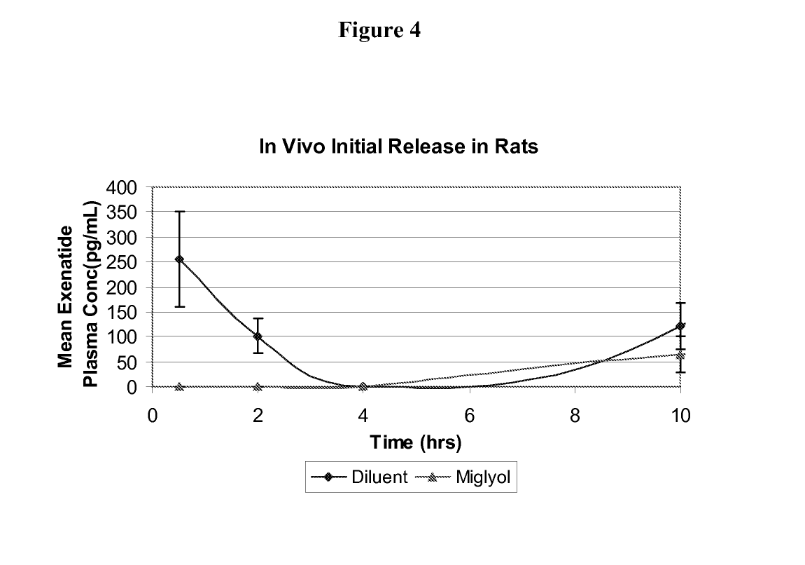

FIG. 4 illustrates the in vivo release profile in rats over 10 hours for the formulation of Example 1 in MCT oil compared to a formulation comprising the same microspheres in an aqueous (saline) carrier. In the time period following sub-cutaneous administration of the formulation, the entrance of exenatide into the plasma was markedly lower than the same microspheres administered in the aqueous carrier. The formulation of the invention shows no burst release, and a markedly more gradual entrance into the blood plasma versus the aqueous formulation. In contrast, the aqueous formulation showed a burst release followed by a sharper entrance into the blood plasma.

### Example 5

Microparticles were prepared in a manner similar to that described in the examples in U.S. Pat. No. 5,439,688, the disclosure of which is incorporated by reference herein. Eight samples were prepared by briefly mixing an active pharmaceutical ingredient (i.e., davalintide, pramlintide, metreleptin, bovine serum albumin, sodium salicylate, salicylic acid, minocycline HCl, insulin) and polymer (i.e, poly(lactide-co-glycolide) copolymer or polycaprolactone/PLGA copolymer) and then the mixture was placed in a grinder to obtain a well-homogenized powder. Mixtures ranged from 2% to 10% w/w of the active pharmaceutical ingredient. The mixed powder was transferred to an extruder where the temperature was adjusted according to the chosen polymer. Some polymers needed higher temperatures to produce a melt with good flow properties. The extruder contained twin screws that moved clockwise to produce efficient mixing. The material was extruded through a 1.5 mm orifice, collected, cooled at room temperature, and cut into short strands about 1-2 inches long. These strands were then fed into a 12-tooth rotor mill, followed by a sieving step to produce microparticles of about 20 to 100 microns. The microparticles were collected and stored at 5° C. until further use.

Experimental samples were prepared by dispersing about 50 mg of the microparticles into 0.75 mL of a MCT oil carrier. The samples were stored at 5° C. and 25° C. for two days, two weeks, or one month, at which times representative samples were tested. The fraction of drug that remained in the microparticles and the fraction of drug that partitioned into the MCT oil carrier were determined. Briefly, the samples were centrifuged to separate the microparticles from the MCT oil carrier. Each portion was treated independently to determine the amount of drug it contained. Results are reported on the basis of the percent residing in each independent portion.

TABLE 4
PLGA copolymer; 2 Days Storage at 5° C.

| Compound | Microparticles | MCT Carrier |
|---|---|---|
| davalintide | 99.8% | 0.2% |
| pramlintide | 100.0% | 0.0% |
| metreleptin | 100.0% | 0.0% |
| bovine serum | 100.0% | 0.0% |
| albumin | | |
| sodium salicylate | 99.5% | 0.5% |
| salicylic acid | 98.9% | 1.1% |
| minocycline | 99.1% | 0.9% |

TABLE 5
PLGA copolymer; 1 Month Storage at 5° C.

| Compound | Microparticles | MCT Carrier |
|---|---|---|
| davalintide | 99.4% | 0.6% |
| pramlintide | 99.7% | 0.3% |
| metreleptin | 100.0% | 0.0% |
| bovine serum | 100.0% | 0.0% |
| albumin | | |
| sodium salicylate | 98.7% | 1.3% |
| salicylic acid | 99.9% | 0.1% |
| minocycline | 99.9% | 0.1% |
| insulin | 99.5% | 0.5% |

TABLE 6
PLGA copolymer; 2 Days Storage at 25° C.

| Compound | Microparticles | MCT Carrier |
|---|---|---|
| davalintide | 100.0% | 0.0% |
| pramlintide | 100.0% | 0.0% |
| metreleptin | 100.0% | 0.0% |
| bovine serum | 100.0% | 0.0% |
| albumin | | |
| sodium salicylate | 97.7% | 2.3% |
| salicylic acid | 99.1% | 0.9% |
| minocycline | 99.4% | 0.6% |

TABLE 7
PLGA copolymer; 1 Month Storage at 25° C.
PLGA Polymer; 1 Month Storage at 25° C.

| Compound | Microparticles | MCT Carrier |
|---|---|---|
| davalintide | 100.0% | 0.0% |
| pramlintide | 100.0% | 0.0% |
| metreleptin | 100.0% | 0.0% |
| bovine serum | 100.0% | 0.0% |
| albumin | | |
| sodium salicylate | 98.5% | 1.5% |
| salicylic acid | 99.8% | 0.2% |
| minocycline | 99.6% | 0.4% |
| insulin | 99.3% | 0.7% |

TABLE 8
polycaprolactone/PLGA copolymer; Two Weeks Storage

| | | | 25° C. | |
|---|---|---|---|---|
| | 5° C. | | | MCT |
| Compound | Microparticles | MCT Carrier | Microparticles | Carrier |
| pramlintide | 100.0% | 0.0% | 100.0% | 0.0% |

The data in Tables 4-8 illustrate the broad applicability of the sustained release formulations described herein to a variety of different active pharmaceutical ingredients, including peptides and small molecules. The compositions have been successfully produced using a variety of peptides, bovine serum albumin, and even a selection of small molecules. Surprisingly salicylic acid, which is oil soluble, did not migrate into the MCT carrier oil, despite that its solubility in the MCT oil is greater than 30 mg/ml. Thus, the microparticles remain intact upon storage in MCT even when the active pharmaceutical ingredient is soluble in MCT. The data further illustrate that the compositions can be successfully produced even using other polymer mixtures in the microparticles.

### Example 6

The percentage purity of exenatide was measured by HPLC at one month intervals over a 9 month period in the following four formulations: (i) a formulation comprising the microspheres of Example 1 stored in an oil MCT oil carrier at 5° C.; (ii) a formulation comprising the microspheres of Example 1 stored in an MCT oil carrier at 25° C.; (iii) dry microspheres of Example 1 that had been stored in a container for 9 months at 5° C. without a liquid carrier, and that were then admixed with an aqueous carrier immediately prior to the study; and (iv) dry microspheres of Example 1 that had been stored in a container for 9 months at 25° C. without a liquid carrier, and that were then admixed with an aqueous carrier immediately prior to the study.

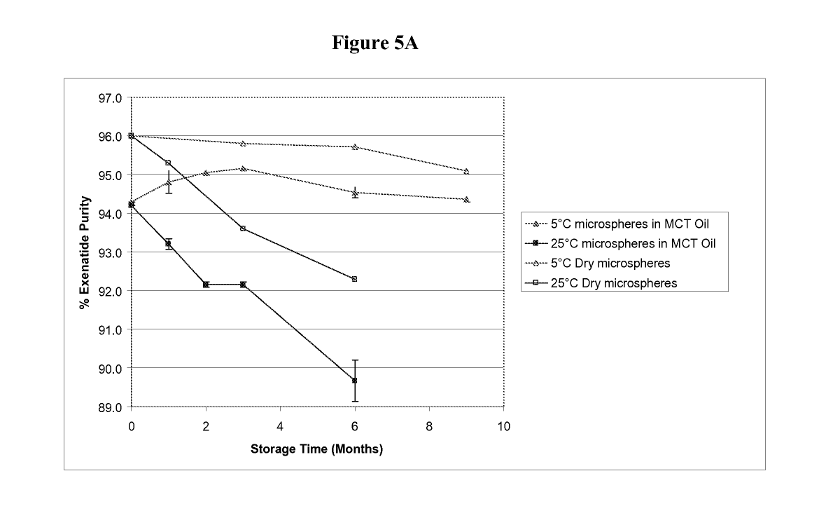

FIGS. 5A and B show the following: (i) exenatide had a purity greater than 93% at 6 months and 9 months in the formulation with the oil carrier at a temperature of 5° C.; (ii) exenatide had a purity greater than 86% at 6 months and 9 months in the formulation with the oil carrier at a temperature of 25° C.; (iii) exenatide had a purity of greater than 94% at 6 months where the microspheres had been stored dry at 5° C.; and (iv) exenatide had a purity of greater than 90% at 6 months in the formulation where the microspheres had been stored dry at a temperature of 25° C. In FIG. 5A, the purity of exenatide was determined by strong cation exchange HPLC. In FIG. 5B, the purity of exenatide was determined by reverse-phase HPLC.

### Example 7

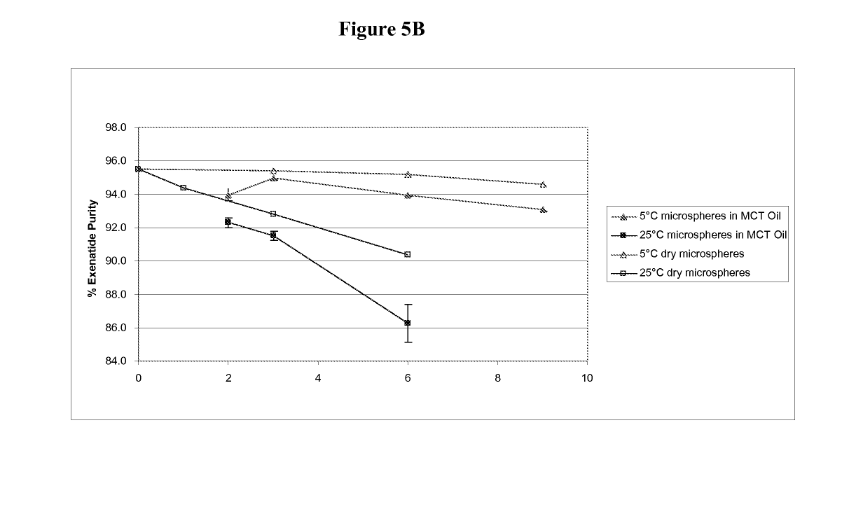

Formulations containing the microspheres from Example 1 and an MCT oil carrier were stored at 5° and the potency of exenatide was measured at monthly intervals for 9 months. Additionally, formulations containing the microspheres from Example 1 and an MCT oil carrier were stored at 25° and the potency of exenatide was measured at monthly intervals for 6 months. FIG. 6 presents the results which show that the potency of exenatide was preserved for at least 9 months.

### Example 8

The physical integrity of a formulation containing the microspheres from Example 1 in an MCT oil carrier was analyzed. After storage for a period of 6 months at 5° C., the molecular weight of the poly(lactide-co-glycolide) copolymer did not change relative to time zero. After storage for a period of 6 months at 25° C., the molecular weight of the poly(lactide-co-glycolide) copolymer decreased by 6 kDaltons, which was comparable to the molecular weight change of dry microspheres (i.e., microspheres stored for 6 months at 25° C. not in any carrier). The mean diameter of the microspheres was measured after storage at 3, 6, and 9 months at either 5° C. or 25° C., and no change in mean diameter was detected relative to time zero.

### Example 9

The ratio of lactide/glycolide for the microparticles was also investigated for use with various APIs. The Table 9 below provides the various lactide/glycolide ratios used.

TABLE 9

| | | Approx. polymer | Lactide/Glycolide |
|---|---|---|---|
| Polymer | Drug | MW (kDa) | ratio for PLGA |
| PLGA | davalintide | 10 | 50/50 |
| PLGA | pramlintide | 10 | 50/50 |
| PLGA | Leptin | 10 | 75/25 |
| PLGA | BSA | 25 | 50/50 |
| PLGA | Na Salicylate | 25 | 50/50 |
| PLGA | Salicylic acid | 25 | 50/50 |
| PLGA | Minocycline | 10 | 75/25 |
| PLGA | Insulin | 25 | 50/50 |
| 1.1:1 | pramlintide | PCL = 150 | 50/50 |
| PCL/PLGA | | PLGA = 10 | |

### Example 10

Exenatide, a GLP-1 receptor agonist, improves glycemic control via multiple mechanisms that stimulate insulin secretion, slowing of gastric emptying, suppression of glucagon secretion, and glucose disposal without increased risk of hypoglycemia. In patients with type 2 diabetes, exenatide is administered as a subcutaneous injection twice daily (BYETTA® by Amylin Pharmaceuticals, Inc. and Eli Lilly and Company); and a once weekly investigational formulation (exenatide once weekly) is currently under FDA review in the United States as a treatment for patients with type 2 diabetes. This once weekly formulation (known as BYDUREON® by Amylin Pharmaceuticals, Inc., Eli Lilly and Company, Alkermes, Inc.) received European marketing authorization from the European Commission in June 2011. A new extended-release formulation (exenatide suspension) utilizes the extended-release microspheres of exenatide once weekly and a medium-chain triglyceride, diluent (described herein) that enables delivery of exenatide with less frequency. Monthly administration of exenatide suspension was investigated in the study described herein.

For purposes of this example, ExQW refers to exenatide once weekly, where the formulation comprised an aqueous suspension of microspheres comprising a poly(lactide-co-glycolide) polymer having dispersed therein about 5% (w/w) exenatide; and about 2% (w/w) sucrose; wherein the ratio of lactide:glycolide in the polymer is about 1:1; and wherein the dosage of exenatide was about 2.0 mg. Once weekly refers to the administration of the formulation to the patients once per week. ExQW will be marketed in the United States, Europe, and throughout the world under the tradename BYDUREON® (Amylin Pharmaceuticals, Inc.; Eli Lilly and Company; Alkermes, Inc.).

For purposes of this example, ExQM refers to exenatide once monthly, where the formulation comprised a non-aqueous suspension of microspheres comprising a poly(lactide-co-glycolide) polymer having dispersed therein about 5% (w/w) exenatide; and about 2% (w/w) sucrose; wherein the ratio of lactide:glycolide in the polymer is about 1:1; and wherein the dosage of exenatide was about 5.0 mg, about 8.0 mg, or about 11.0 mg. Once monthly refers to the administration of the formulation to the patients once per month (e.g., once every four weeks). The non-aqueous suspension comprised the microspheres in MIGLYOL® 812 (Sasol Germany GmbH, Witten, Germany), a medium chain triglyceride.

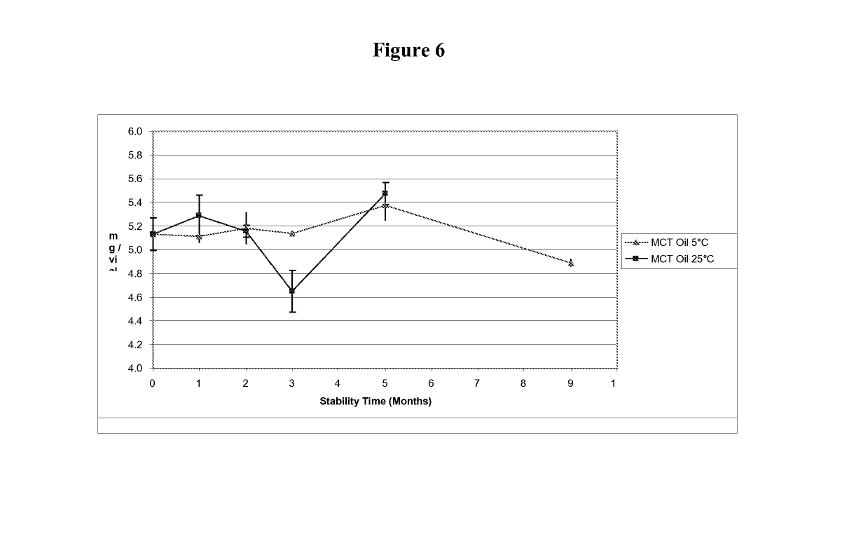

A Phase 2, randomized, controlled, feasibility study with blinded ExQM doses (shown in FIG. 7) was undertaken. Beginning at day 1, subjects received either (a) 20 weekly injections of ExQW (2 mg) with the last injection at week 19; (b) 5 monthly injections of ExQM 5.0 mg with the last injection at week 16; (c) 5 monthly injections of ExQM 8.0 mg with the last injection at week 16; or (d) 5 monthly injections of ExQM 11.0 mg with the last injection at week 16.

Main Inclusion Criteria for patients: Patients at least 18 years of age with type 2 diabetes treated with diet/exercise, metformin (MET), pioglitazone (PIO), or metformin+pioglitazone (MET+PIO) for a minimum of 2 months at screening; A1C of 7.1% to 11.0% (inclusive); fasting plasma glucose (FPG) less than 280 mg/dL; and stable body weight (not varying by more than 3% for at least 3 months prior to screening).

The Intent-To-Treat (ITT) Population: N=121: all subjects who received at least 1 dose of study medication. The Evaluable Population: N=110: all subjects who completed study procedures through 20 weeks in compliance with the protocol and received adequate study medication exposure. The Pharmacokinetic (PK) Evaluable Population N=99: all ITT subjects with ≧half of PK samples>lower level of detection.

Statistical Analysis: The study was powered to ensure an 80% probability of observing ≧0.9% A1C reduction from baseline to week 20 for any ExQM treatment group. The primary endpoint was the change in A1C from baseline (day 1) to week 20; safety, PK, and pharmacodynamics were monitored for an additional 4 weeks. Superpositioning techniques using 10 mg single dose exenatide suspension data were employed to predict the ExQM doses and PK disposition. Descriptive statistics for the Evaluable Population were calculated for efficacy (A1C, FPG, and body weight) and PK parameters by treatment and time; evaluable and ITT results were comparable. Descriptive statistics for the ITT population were calculated for demographics and safety endpoints by treatment; adverse events were reported as overall incidence in the ITT population.

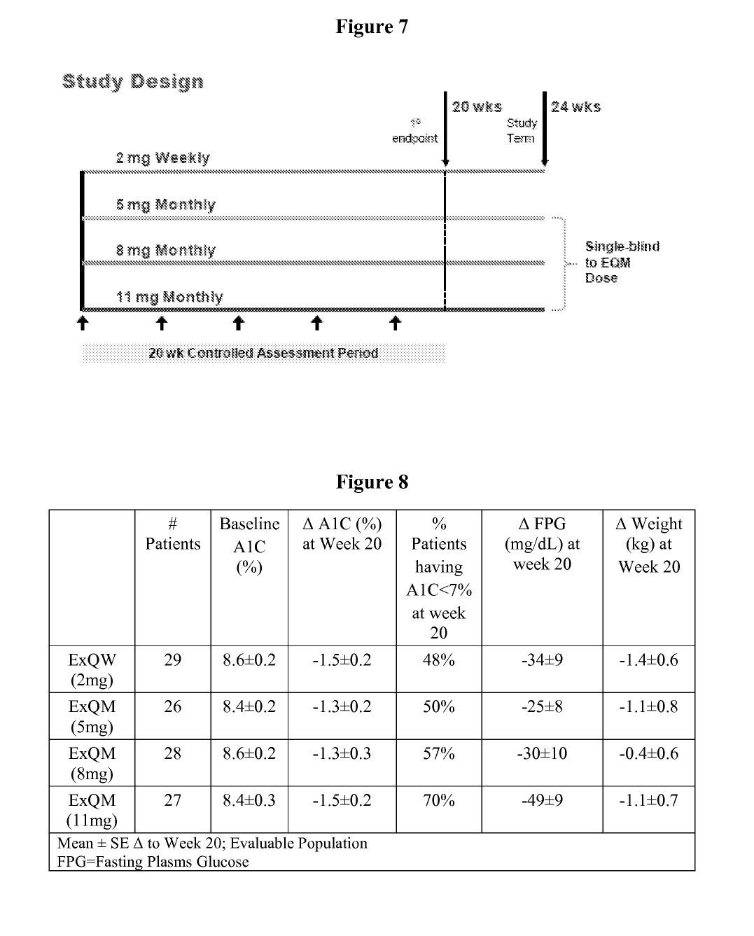

After 20 weeks of treatment, the results for ExQW (2 mg) and ExQM (5 mg, 8 mg, 11 mg) with respect to the patients' change in A1C, percentage of patients achieving an A1C of less than 7%; patients' change in fasting plasma glucose (FPG); and patients' change in body weight are presented in the Table at FIG. 8.

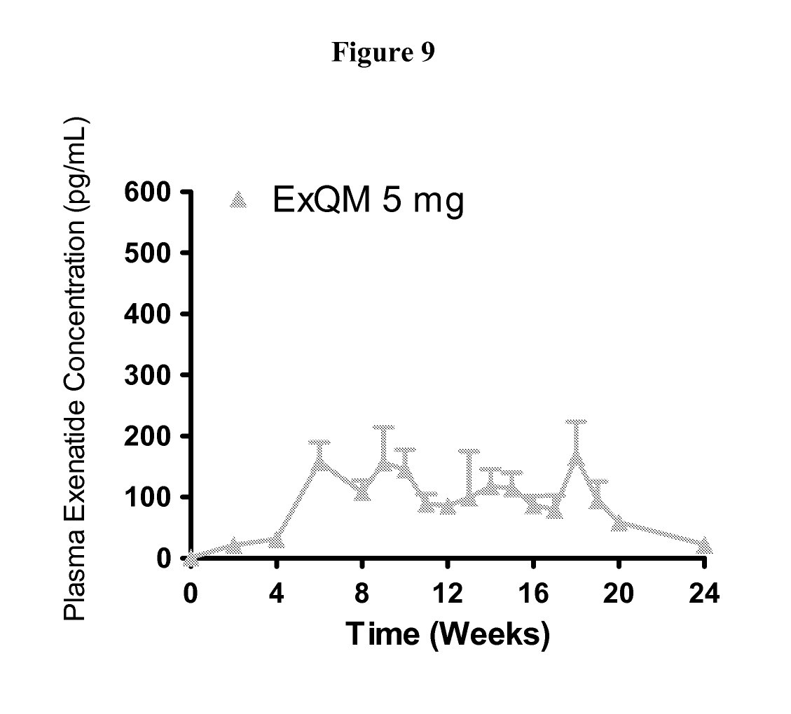

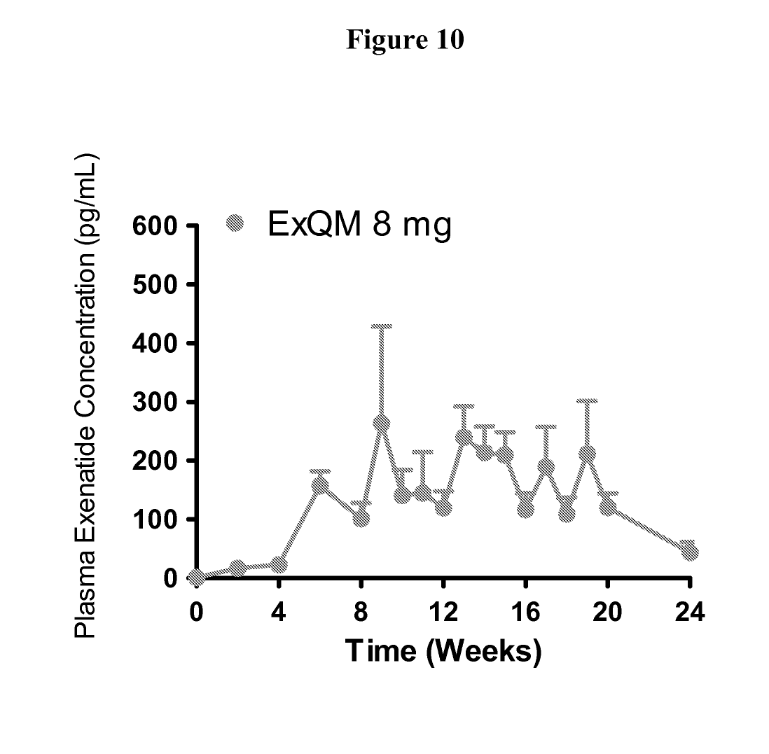

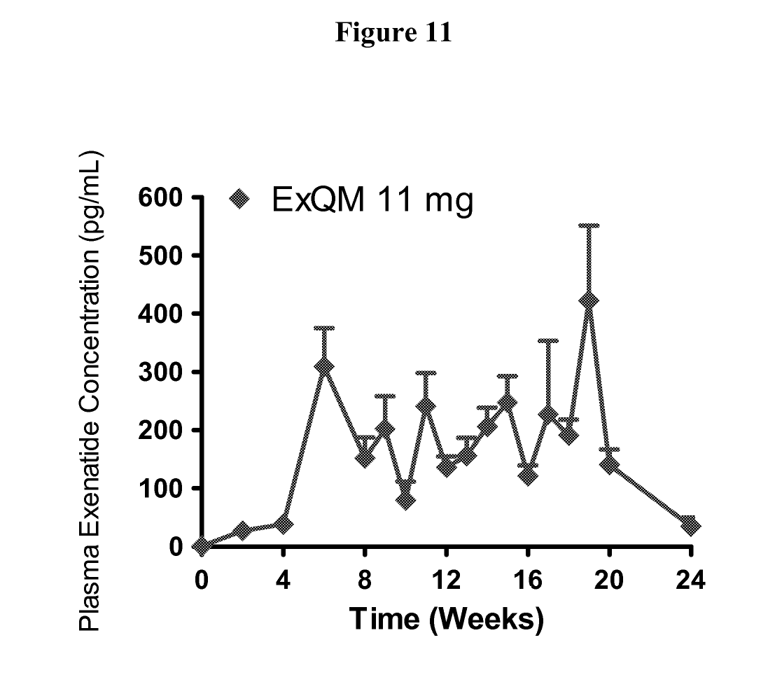

Pharmacockintic (pK) results shown for ExQM 5 mg, 8 mg, and 11 mg are shown in FIGS. 9-11, respectively. Observed exposure with 5 mg and 8 mg ExQM closely replicated predicted values; and slightly lower concentrations were achieved with 11 mg ExQM. Monthly dosing resulted in minimal accumulation and greater peak-to-trough ratios compared to ExQW; and mean trough concentrations for all ExQM doses remained within the therapeutic range, even at doses as low as 5 mg. The two highest ExQM doses achieved maximum concentrations similar to ExQW. As established previously with ExQW, ExQM approached undetectable levels 8 wks after last injection. pK data for ExQW is publically available at, e.g., US Publication No. 2009/0239796, the disclosure of which is incorporated by reference herein.

Monthly administration of exenatide suspension by subcutaneous injection at doses of 5 mg, 8 mg, and 11 mg for 20 weeks was safe and well-tolerated in patients with type 2 diabetes. No unique safety findings were observed with ExQM relative to ExQW. The mean pharmacokinetic profile showed sustained plasma exenatide concentrations with trough concentrations above minimally effective exenatide concentrations. Substantial reductions in A1C and FPG were observed with ExQW and all doses of ExQM. The range of weight loss observed with ExQM was similar to that seen with ExQW.

### Example 11

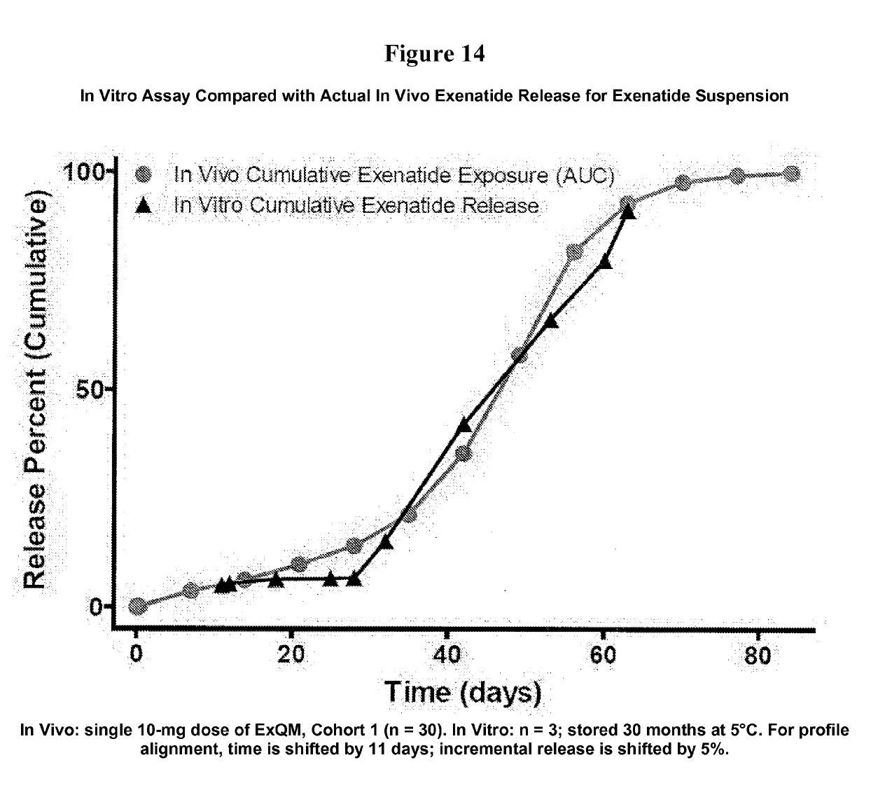

Using an in vitro method to compare release profiles, exenatide suspension demonstrated a blunted initial release compared to aqueous ExQW. FIGS. 12-14 compare the in vitro release profile with the in vivo release profile. For the in vitro analysis, samples of a known quantity were incubated in Tris-buffered medium (pH 9.4) at 37° C.; aliquots of medium were removed on set sampling days. The exenatide concentration in the medium was determined by size exclusion HPLC with external standard calibration by UV absorbance.

For purposes of this example, ExQW refers to exenatide once weekly, where the formulation comprised an aqueous suspension of microspheres comprising a poly(lactide-co-glycolide) polymer having dispersed therein about 5% (w/w) exenatide; and about 2% (w/w) sucrose; wherein the ratio of lactide:glycolide in the polymer is about 1:1; and wherein the dosage of exenatide was about 2.0 mg. Once weekly refers to the administration of the formulation to the patients once per week. ExQW will be marketed in the United States, Europe, and throughout the world under the tradename BYDUREON® (Amylin Pharmaceuticals, Inc.; Eli Lilly and Company; Alkermes, Inc.).

For purposes of this example, ExQM refers to exenatide once monthly, where the formulation comprised a non-aqueous suspension of microspheres comprising a poly(lactide-co-glycolide) polymer having dispersed therein about 5% (w/w) exenatide; and about 2% (w/w) sucrose; wherein the ratio of lactide:glycolide in the polymer is about 1:1; and wherein the dosage of exenatide was 10.0 mg. Once monthly refers to the administration of the formulation to the patients once per month (e.g., once every four weeks). The non-aqueous suspension comprised the microspheres in MIGLYOL® 812 (Sasol Germany GmbH, Witten, Germany), a medium chain triglyceride.

Cohort 1 included healthy patients 19-65 years old with a body mass index of 23 kg/m2 to 35 kg/m2 and no history of diabetes. A single 10-mg dose in 30 healthy volunteers confirmed the in vitro profile; exenatide concentrations increased gradually over time, peaked at wk 6-7 and approached lower level of detection after ˜wk 10.

Cohort 2 included patients 19-75 years old with T2DM treated with diet/exercise alone, or combination of MET and/or PIO for a minimum of 2 months at screening. They had an A1C of 7.1% to 10.0%; fasting plasma glucose (FPG)<260 mg/dL; and a body mass index 25 kg/m2 to 45 kg/m2. Thirty-five (35) patients with type II diabetes mellitus (T2DM) (31% F, 52±11 y, WT 105±22 kg, A1C 8.0±0.9%, FPG 167±34 mg/dL, mean±SD) treated with diet/exercise, metformin (MET), pioglitazone (PIO), or MET+PIO were randomized to exenatide once weekly suspension (EQWS 2 mg SC, N=23) or MCT control (SC, N=12). EQWS achieved mean Css by ˜wk 8 that were in the range of Css previously seen with ExQW. At wk 12, LS mean [SE] change from baseline was significantly greater with EQWS than MCT for A1C (−0.9 [0.2] vs +0.1 [0.2]%; P=0.0013) and FPG (−32 [10] vs +8 [12] mg/dL, P=0.0035) and was associated with weight loss (−1.4 kg).

No unique safety findings were observed with EQWS relative to ExQW. EQWS was well tolerated, with improvements in glycemic control and weight loss in patients with T2DM comparable to ExQW, supporting further development of EQWS. The treatment-emergent adverse events with incidence ≧10% are presented in the Table 10 below.

TABLE 10
Treatment-Emergent Adverse Events with Incidence ≧10% in
Any Treatment Arm (EQWS or MCT-only control)

| | Cohort 1 | Cohort 2 | |
|---|---|---|---|
| | Single 10-mg | | MCT-only |
| | dose EQWS | EQWS 2 mg | Control |
| | (N = 30) | (N = 23) | (N = 12) |
| | n (%) | n (%) | n (%) |
| Injection-site arythema | 4 (13.3) | 12 (52.2) | 1 (8.3) |
| Injection-site pruritus | 10 (33.3) | 11 (47.8) | 1 (8.3) |
| Injection-site haematoma | 5 (16.7) | 9 (39.1) | 6 (50.0) |
| Nauses | 3 (10.0) | 5 (21.7) | 1 (8.3) |
| Decreased appatite | 3 (10.0) | 5 (21.7) | 0 (0.0) |
| Injection-site pain | 8 (20.0) | 5 (21.7) | 0 (0.0) |
| Diarrhea | 0 (0.0) | 4 (17.4) | 2 (16.7) |
| Headache | 6 (20.0) | 3 (13.0) | 0 (0.0) |
| Pruritus | 0 (0.0) | 3 (13.0) | 0 (0.0) |
| Oropharyngeal pain | 3 (10.0) | 1 (4.3) | 0 (0.0) |

As shown by the Table 10 above, Cohort 1 EQWS 10 mg (once monthly per the present disclosure) was well tolerated, with the majority of treatment-emergent adverse events of mild intensity. No major or minor hypoglycemia was observed. Improvements in mean systolic blood pressure were observed; mean diastolic pressure increased with active treatment to a similar extent as MCT-only control. Antibodies to exenatide results with EQWS 10 mg were consistent with what has been observed with EQW. Other than increased incidence of injection-site reactions, adverse event rates were similar in antibody-positive and antibody-negative patients.

### Example 12

A population PK/PD simulation model was designed to estimate the effects of ExQM in a 28 week treatment period. All simulations were performed using a population pharmacokinetic (PK) and pharmacokinetic/pharmacodynamic (PK/PD) model for once-monthly exenatide suspension (EQMS) based on data from two clinical trials: a trial with subjects given a single 10 mg dose of exenatide suspension (Example 11; Cohort 1) and a trial with subjects given monthly doses of exenatide suspension at 5, 8 or 11 mg (Example 10).

Apparent disposition-related population parameters (clearance [CL/F], central volume [VC/F], inter-compartmental clearance [Q/F] and peripheral volume [VP/F]) from previous BYETTA model development were held fixed to aid in the model fitting of exenatide suspension data. To account for potential differences in the bioavailability of exenatide suspension compared to BYETTA, a relative bioavailability term (FE) was included.

In order to describe the non-linear absorption observed with the release of exenatide from microspheres in suspension, a simultaneous dual absorption of a transit-compartment depot mechanism and a zero-order infusion were used. Exenatide once monthly suspension data from Example 10 showed a lack of dose proportionality. Therefore, bioavailability was incorporated into the PK model with a linear function relating relative bioavailability (FE) to dose concentration (D_{CONC}).

Functions to describe how population characteristics (covariates) influenced disposition related parameters (CL/F, VC/F, Q/F and VP/F) were fixed to parameters previously identified in BYETTA model development; however, the influence of antibody titer (TITR) on apparent clearance (CL/F) was estimated using EQMS data. Additionally, baseline body weight (WTKB) and subject age (AGEY) were included as covariates on the transit compartment transfer rate constant (KTR).

The population PK/PD model incorporated a direct effect between exenatide plasma concentration and fasting plasma glucose (FPG) levels using an EMAX model. The rate of HbA1c appearance was modeled with a rate constant, proportional to FPG levels. Baseline FPG and HbA1c levels were estimated, and the system was assumed to be at equilibrium prior to the first exenatide dose administration.

For the population PK and population PK/PD models, exponential functions were used for all inter-individual variability terms and residual variability was modeled using proportional error.

All population PK and PK/PD models were developed and all simulations were performed using NONMEM software, Version VI, Level 2.0, with NM-TRAN, Version IV, Level 1.0 and PREDPP, Version V, Level 1.0. NONMEM analyses were performed on a Microsoft Windows-based Intel cluster with the Windows Server 2003 Enterprise Edition operating system. The Fortran compiler used was Intel Visual FORTRAN 10.1.032.

The first table below, Table 11, shows the estimated parameters for the population PK model and the subsequent table shows the estimated parameters for the population PK/PD model used in simulations.

TABLE 11
Estimated Population PK Parameters for Combined
Single and Multiple Dose Data

| | | Standard Error |
|---|---|---|
| Parameter | Estimate | of Estimate (% CV) |
| KTR (1/hr) | 0.0385 | 10.9 |
| D1 (hr) | 1460 | 2.90 |
| Finf | 0.323 | 6.75 |
| FE (D_{CONC} = 5.56 mg/mL) | 0.168 | 6.31 |
| Slope (FE vs D_{CONC}) | 0.00323 | 88.2 |
| NN | 43.6 | 11.1 |
| CL/F (L/hr) | 7.65 FIXED | — |
| VC/F (L) | 13.0 FIXED | — |
| Q/F (L/hr) | 1.81 FIXED | — |
| VP/F (L) | 103 FIXED | — |
| TITR on CL/F (TITR>125) | −2.27 | 62.1 |
| WTKB on KTR (power) | −0.461 | 20.7 |
| AGEY on KTR (linear) | −0.175 | 29.0 |
| CRCL on CL (linear) | 0.0533 FIXED | — |
| WTKB on CL (linear) | 0.0723 FIXED | — |
| WTKB on VC (exponential) | 0.0140 FIXED | — |
| ω_{KTR} (% CV) | 7.91 | 41.5 |
| ω_{CL/F} (% CV) | 32.2 | 31.6 |
| ω_{VC/F} (% CV) | 58.2 | 55.5 |
| ω_{Q/F (% CV)} | 126 | 35.3 |
| σ (% CV) for Example 11 | 34.6 | 9.83 |
| σ (% CV) for Example 10 | 73.4 | 7.87 |

*MTT (Computed as NN + 1/KTR) = 6.86 weeks.
Definitions:
KTR is the transfer rate constant of exenatide between each transit compartment, in 1/hr
D1 is the duration of the modeled infusion administration, in hr
Finf is the fraction of the i^{th} dose administered as infusion
D_{CONCi} is the dose concentration of the i^{th} formulation, in mg/mL
FE is the relative bioavailability of exenatide suspension compared to BYETTA for
COHORT 1 (D_{CONC} = 5.56 mg/mL)
Slope is the estimated slope relating relative bioavailability (FE) and concentration of exenatide in the dose (D_{CONC})
NN is the number of the last transit compartment, which leads into the depot compartment
CL/F is the apparent clearance from the central compartment, in L/hr
VC/F is the apparent volume of the central compartment, in L
Q/F is the apparent inter-compartmental clearance rate, between central and peripheral compartments, in L/hr
VP/F is the apparent volume of the peripheral compartment, in L
TITR is the antibody to exenatide titer level
WTKB is the baseline body weight, in kg
AGEY is the subject's age, in yr
CRCL is creatinine clearance, in mL/min
ωi is the between-subject variability of i^{th} parameter
CV is the coefficient of variation
σ is the residual variability

TABLE 12
Estimated Population PK/PD Parameters

| | | Standard Error of |
|---|---|---|
| Parameter | Estimate | Estimate(% CV) |
| KD (1/Day) | 0.0327 | 16.9 |
| EMAX | 0.278 | 10.5 |
| EC50 (pg/mL) | 114 | 26.0 |
| BSFG (mg/dL) | 168 | 2.76 |
| BSA1 (%) | 8.40 | 1.63 |
| ω_{KD} (% CV) | 78.4 | 35.4 |
| ω_{EMAX} (% CV) | 47.2 | 33.1 |
| ω_{EC50} (% CV) | 95.2 | 52.0 |
| ω_{BSFG} (% CV) | 23.5 | 12.8 |
| ω_{BSA1} (% CV) | 13.1 | 11.5 |
| CORRω_{KD,} ω_{EMAX} | −91.0 | 30.9 |
| CORRω_{BSFG,} ω_{BSA1} | 87.2 | 12.6 |
| σ_{BSFG} (% CV) | 14.5 | 9.72 |
| σ_{BSA1} (% CV) | 4.69 | 17.9 |

Definitions:
KD is the rate of FPG regulated appearance of HbA1c, in %/mg/dL/hr
EMAX is the maximum effect on glucose by exenatide
EC50 is the exenatide concentration that causes half of the maximum effect, in pg/mL
BSFG is the baseline level of FPG prior to exposure to exenatide, in mg/dL
BSA1 is the baseline level of HbA1c prior to exposure to exenatide, in %
CORR_{ωi,ωj} is the correlation coefficient between the variances of parameter i and parameter j

The proposed range of doses for further evaluation was chosen based on target glycemic response (HbA1c, fasting and postprandial glucose), percentage of time spent above target concentration predicted to impact weight loss, and steady-state average exposure consistent with the historical target exposures.

Target Reduction in HbA1c

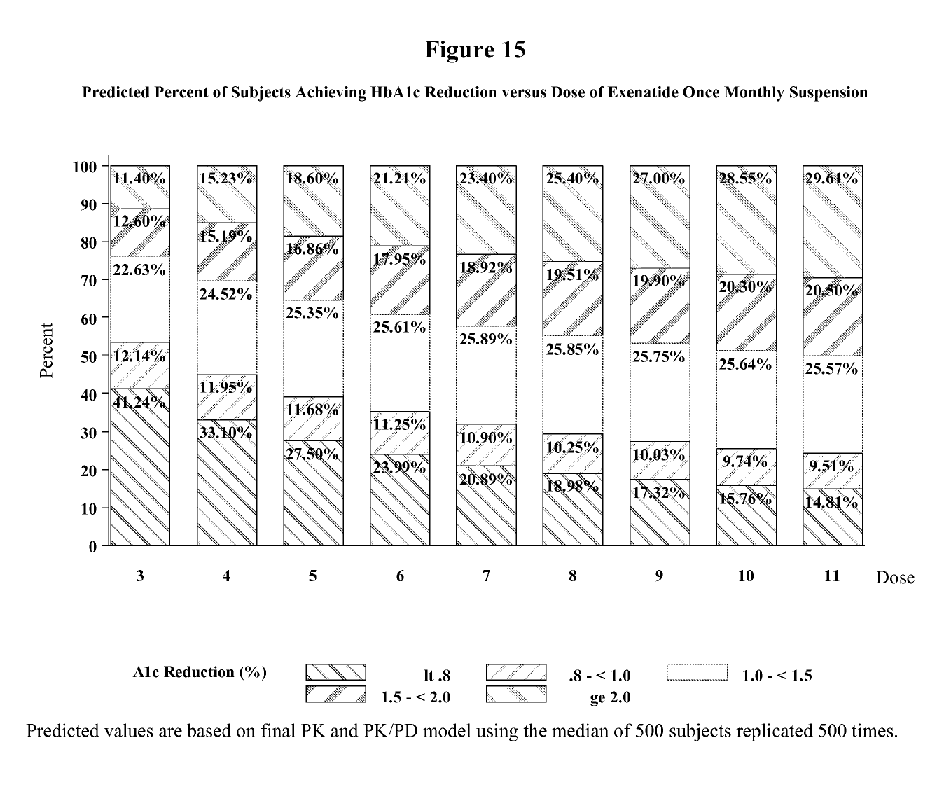

Simulations were performed to determine the percentage of subjects predicted to reach an end of trial change in HbA1c from baseline of at least −1.5%, similar to the efficacy typically observed with exenatide once weekly. Using the final PK/PD model and a range of simulated doses from 3 mg to 11 mg exenatide once monthly suspension, the change in HbA1c was calculated for a 28-week treatment period based on the above evaluation of study duration. In order to assess the robustness of the PK/PD response across the simulated doses, the change in HbA1c levels were divided into 5 categories: <0.8%, 0.8% to <1%, 1% to <1.5%, 1.5% to <2%, and ≧2%. The percentage of subjects at each change in HbA1c category was calculated for each dose. This analysis demonstrated that doses of 8 mg and greater are predicted to result in at least 45% of the subjects achieving a change in HbA1c of at least 1.5%. This is graphically shown in FIG. 15.

Achieving Steady-State Average Exposures

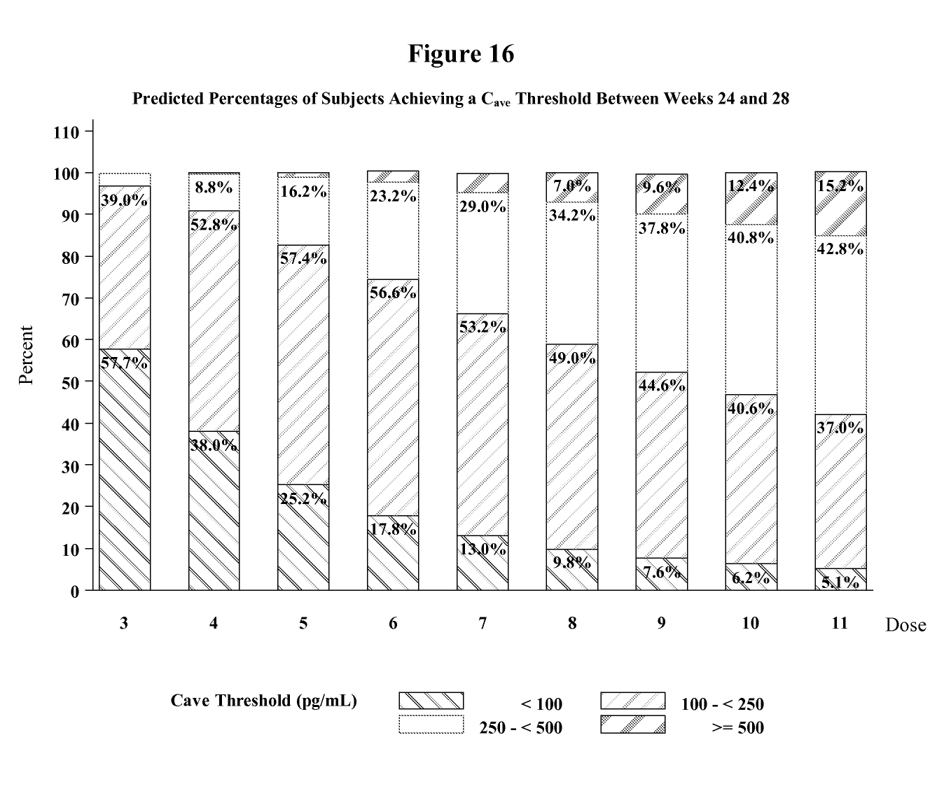

Target concentrations of exenatide associated with a robust HbA1c response fall within the range of 200 to 300 pg/mL, the Cₘₐₓ of BYETTA is 211 pg/mL, and the steady-state average exposure of exenatide once weekly from a pooled analysis of subjects with varying levels of renal function is 302 pg/mL. Simulations suggest that doses of 9 mg exenatide once monthly suspension or higher will result in approximately 45% of the subjects achieving a Cₐᵥₑ concentration of at least 250 pg/ml, which reflects an exposure in the upper half of the target concentration range for exenatide. This is graphically shown in FIG. 16.

The Percentage of Time Spent Above Target Concentrations Believed to Impact Centrally Mediated Effects (Weight Loss)

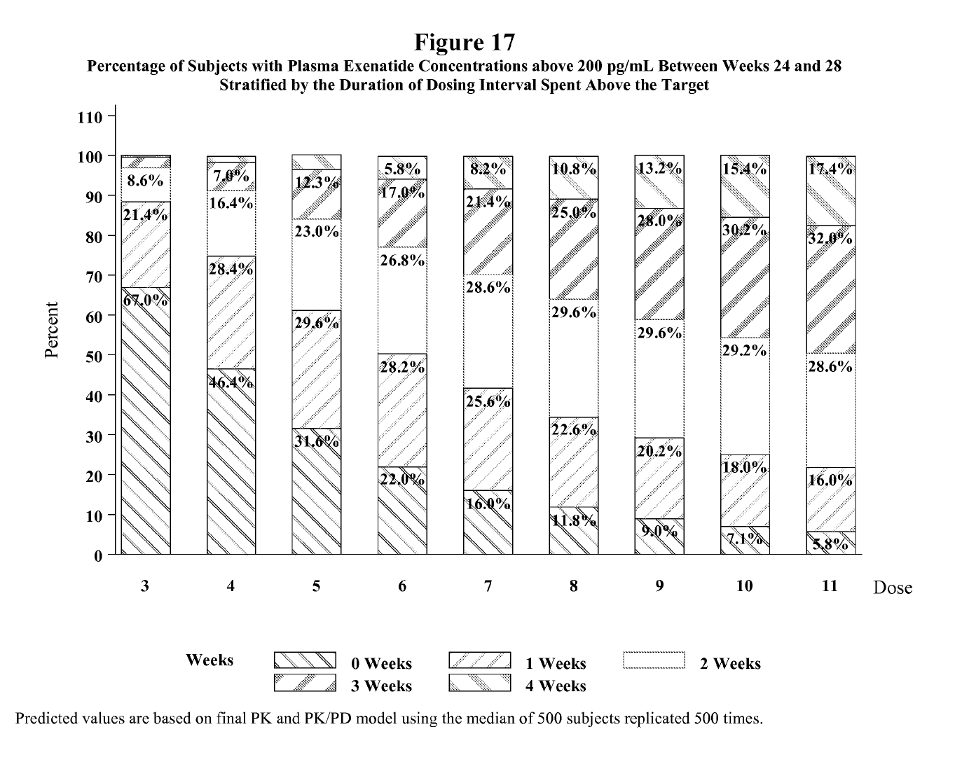

In a previously developed PK/PD model for exenatide once weekly extended release (aqueous diluent), the estimated EC₅₀ for exenatide was 56.8 pg/mL and 184 pg/mL for FPG reduction and body weight loss, respectively. These findings suggest that higher plasma exenatide concentrations are required to elicit weight loss. Therefore, a dose of exenatide once monthly suspension was considered preferable when the majority of subjects had an average plasma exenatide concentration of 200 pg/mL or greater for more than half the dosing interval (i.e. 2 weeks). FIG. 17 shows that doses greater than 9 mg are predicted to have approximately 70% of subjects above 200 pg/mL for more than half of the dosing interval (2 weeks). Doses of 7 mg and 8 mg also achieved the desired average plasma exenatide concentration or greater for more than half the dosing interval.

### Range of Doses

When considering the relationship between dose, response, and exposure, it is important to select a dose or doses that balances the risk of potential adverse events and still provides a robust efficacious response. While the number of adverse events in Example 10 was too small to formally assess an exposure-response relationship, a slight trend was observed for an increased incidence of adverse events (e.g. nausea and vomiting) at the highest dose (11 mg) compared to the 2 lower doses (5 and 8 mg). Taken together with the simulations described above that identified dose ranges sufficient to elicit robust glycemic improvements, weight loss, and exenatide exposures consistent with known therapeutic ranges, doses of exenatide once monthly suspension in the range of 6 mg to 10 mg, 7 mg to 9 mg, about 7 mg, about 8 mg, or about 9 mg are believed to be superior.

Doses in the range of 6 mg to 10 mg, more preferably 7 mg to 9 mg, preferably about 7 mg, about 8 mg, or about 9 mg, of exenatide once monthly suspension are predicted to result in a robust reduction in HbA1c, a robust reduction in body weight loss, while reducing FPG, with similar exposure levels to historical exenatide concentrations, while minimizing the potential gastrointestinal adverse events. A dose at the higher end of the range may be investigated to maximize effects on glycemic control and body weight. For a proposed Phase 3 clinical trial, the applicant has chosen to evaluate single 7 mg and 9 mg doses administered once monthly.

All publications and patents are incorporated by reference herein. The foregoing has been described in detail, and the skilled artisan will recognize that modifications may be made without departing from the spirit or scope of the disclosure or appended claims.

1-51. (canceled)

52. A manufactured pre-mixed formulation for injection comprising a suspension of
(i) a pharmaceutically acceptable non-aqueous carrier; and
(ii) microspheres which comprise a biocompatible, biodegradable polymer and a GLP-1 receptor agonist present in an amount of 3 mg to 12 mg.

53-82. (canceled)

83. The formulation of claim 52, wherein the GLP-1 receptor agonist is present in an amount of 7.5 mg to 12 mg.

84. The formulation of claim 52, wherein the GLP-1 receptor agonist is present in an amount of 6 mg to 10 mg.

85-86. (canceled)

87. The formulation of claim 52, wherein the GLP-1 receptor agonist is present in an amount of 8 mg.

88. The formulation of claim 52, wherein the GLP-1 receptor agonist is present in an amount of 9 mg.

89. (canceled)

90. The formulation of claim 52, wherein the GLP-1 receptor agonist is exendin-4.

91-92. (canceled)

93. The formulation of claim 52, wherein the GLP-1 receptor agonist is exendin-4, Leu¹⁴-exendin-4 (SEQ ID NO: 3); Leu¹⁴,Phe²⁵-exendin-4 (SEQ ID NO: 4); Leu¹⁴,Ala¹⁹,Phe²⁵-exendin-4 (SEQ ID NO: 5); exendin-4(1-30) (SEQ ID NO: 6); Leu¹⁴-exendin-4(1-30) (SEQ ID NO: 7); Leu¹⁴,Phe²⁵-exendin-4(1-30) (SEQ ID NO: 8); Leu¹⁴,Ala¹⁹,Phe²⁵-exendin-4(1-30) (SEQ ID NO: 9); exendin-4(1-28) (SEQ ID NO: 10); Leu¹⁴-exendin-4(1-28) (SEQ ID NO: 11); Leu¹⁴,Phe²⁵-exendin-4(1-28) (SEQ ID NO: 12); Leu¹⁴,Ala¹⁹,Phe²⁵-exendin-4 (1-28) (SEQ ID NO: 13); Leu¹⁴,Lys^{17,20},Ala¹⁹,Glu²¹,Phe²⁵,Gln²⁸-exendin-4 (SEQ ID NO: 14); Leu¹⁴,Lys^{17,20},Ala¹⁹,Glu²¹,Gln²⁸-exendin-4 (SEQ ID NO: 15); octylGly¹⁴,Gln²⁸-exendin-4 (SEQ ID NO: 16); Leu¹⁴,Gln²⁸,octylGly³⁴-exendin-4 (SEQ ID NO: 17); Phe⁴,Leu¹⁴,Gln²⁸,Lys³³,Glu³⁴, Ile^{35,36},Ser³⁷-exendin-4(1-37) (SEQ ID NO: 18); Phe⁴,Leu¹⁴,Lys^{17,20},Ala¹⁹,Glu²¹,Gln²⁸-exendin-4 (SEQ ID NO: 19); Val¹¹,Ile¹³,Leu¹⁴,Ala¹⁶,Lys²¹,Phe²⁵-exendin-4 (SEQ ID NO: 20); exendin-4-Lys⁴° (SEQ ID NO: 21); lixisenatide; CJC-1134; [N^{e}-(17-carboxyheptadecanoic acid)Lys²⁰]exendin-4-NH₂ (SEQ ID NO: 46); [N^{e}-(17-carboxyhepta-decanoyl)Lys³²]exendin-4-NH₂ (SEQ ID NO: 47); [desamino-His¹,N^{e}-(17-carboxyheptadecanoyl)Lys²⁰]exendin-4-NH₂ (SEQ ID NO: 48); [Arg^{12,27},NLe¹⁴,N^{e}-(17-carboxy-heptadecanoyl)Lys³²]exendin-4-NH₂ (SEQ ID NO: 49); [N^{e}-(19-carboxy-nonadecanoylamino)Lys²⁰]-exendin-4-NH₂ (SEQ ID NO: 50); [N^{e}-(15-carboxypentadecanoylamino)Lys²⁰]-exendin-4-NH₂ (SEQ ID NO: 51); [N^{e}-(13-carboxytridecanoylamino)Lys²⁰]exendin-4-NH₂ (SEQ ID NO: 52); [N^{e}-(11-carboxy-undecanoyl-amino)Lys²⁰]exendin-4-NH₂ (SEQ ID NO: 53); exendin-4-Lys⁴⁰(e-MPA)-NH₂ (SEQ ID NO: 54); exendin-4-Lys⁴⁰(e-AEEA-AEEA-MPA)-NH₂ (SEQ ID NO: 55); exendin-4-Lys⁴⁰(e-AEEA-MPA)-NH₂ (SEQ ID NO: 56); exendin-4-Lys⁴⁰(e-MPA)-albumin (SEQ ID NO: 57); exendin-4-Lys⁴⁰(e-AEEA-AEEA-MPA)-albumin (SEQ ID NO: 58); or exendin-4-Lys⁴⁰(e-AEEA-MPA)-albumin (SEQ ID NO: 59).

94-99. (canceled)

100. The formulation of claim 52, wherein the microspheres further comprise a sugar.

101-105. (canceled)

106. The formulation of claim 52, wherein the pharmaceutically acceptable non-aqueous carrier comprises one or more triglycerides.

107-111. (canceled)

112. The formulation of claim 106, wherein the one or more triglycerides comprise (i) 0 to 2 wt % C₆ fatty acid, 65 to 80 wt % C₈ fatty acid, 20 to 35 wt % C₁₀ fatty acid, and 0 to 2 wt % C₁₂ fatty acid; (ii) 0 to 2 wt % C₆ fatty acid, 50 to 65 wt % C₈ fatty acid, 30 to 45 wt % C₁₀ fatty acid, and 0 to 2 wt % C₁₂ fatty acid; (iii) 0 to 2 wt % C₆ fatty acid, 45 to 65 wt % C₈ fatty acid, 30 to 45 wt % C₁₀ fatty acid, 0 to 3 wt % C₁₂ fatty acid; and 0 to 5 wt % linoleic acid; or (iv) 0 to 2 wt % C₆ fatty acid, 45 to 55 wt % C₈ fatty acid, 30 to 40 wt % C₁₀ fatty acid, 0 to 3 wt % C₁₂ fatty acid, and 10 to 20 succinic acid.

113. (canceled)

114. The formulation of claim 112, wherein the one or more triglycerides comprise 0 to 2 wt % C₆ fatty acid, 50 to 65 wt % C₈ fatty acid, 30 to 45 wt % C₁₀ fatty acid, and 0 to 2 wt % C₁₂ fatty acid.

115. (canceled)

116. The formulation of claim 52, wherein the biocompatible, biodegradable polymer is a poly(lactide-co-glycolide) copolymer.

117-123. (canceled)

124. A method for treating diabetes, for treating overweight, for treating obesity, for reducing body weight, for treating a cardiovascular disease, for treating fatty liver, for treating a gastrointestinal disease, or for treating a neurodegenerative disease in a patient in need thereof, the method comprising
administering to the patient the formulation of claim 52 to treat diabetes, to treat overweight, to treat obesity, to reduce body weight, to treat a cardiovascular disease, to treat fatty liver disease, to treat a gastrointestinal disease, or to treat a neurodegenerative disease.

125. The method of claim 124, wherein the formulation is administered to the patient once a month.

126. The method of claim 124, wherein the formulation is administered to the patient once every four weeks.

127-138. (canceled)

139. The method of claim 124, wherein the formulation achieves a therapeutically effective mean steady state plasma concentration of the GLP-1 receptor agonist in the subject of 170 pg/ml to 330 pg/ml for at least one month.

140-173. (canceled)

174. The method of claim 124, wherein the administration of the formulation achieves an in vivo release profile having a small transient rise over the first 8 hours, followed by a plateau, and one large peak at about 6-7 weeks, wherein about 70% of exenatide or the GLP-1 receptor agonist is released between weeks 4 and 8, the Tₘₐₓ occurs in the large peak at about 42-49 days, and less than 0.5% of the exenatide or the GLP-1 receptor agonist is released within the first 24 hours after injection.

175-177. (canceled)

178. The method of claim 124 for treating diabetes, the method comprising monthly dosing of the formulation,
wherein the administration of an initial dose of the formulation achieves an in vivo release profile having a small transient rise over the first 8 hours, followed by a plateau, wherein less than 0.5% of the GLP1 receptor agonist is released within the first 24 hours;
and wherein the in vivo release profile at steady state has the following characteristics:
(i) the maximum plasma concentration is achieved at approximately 2 weeks after each monthly dose;
(ii) the peak to trough ratio following each monthly dose ranges between 5 to 9, or is about 5, 6, 7, 8, or 9.

179-201. (canceled)

202. A container comprising the formulation of claim 52.

203. The container of claim 202, wherein the container is a pen injector, a vial, or a cartridge.


SEQUENCE LISTING (64 sequences)

SEQ ID NO: 1 - protein, 39 aa, Heloderma suspectum
  C-term amidated
HGEGTFTSDLSKQMEEEAVRLFIEWLKNGGPSSGAPPPS

SEQ ID NO: 2 - protein, 39 aa, Heloderma horridum
  C-term amidated
HSDGTFTSDLSKQMEEEAVRLFIEWLKNGGPSSGAPPPS

SEQ ID NO: 3 - protein, 39 aa, Homo sapiens
  C-term may or may not be amidated
HGEGTFTSDLSKQLEEEAVRLFIEWLKNGGPSSGAPPPS

SEQ ID NO: 4 - protein, 39 aa, Artificial Sequence
  Description of Artificial Sequence Synthetic polypeptide
HGEGTFTSDLSKQLEEEAVRLFIEFLKNGGPSSGAPPPS

SEQ ID NO: 5 - protein, 39 aa, Artificial Sequence
  Description of Artificial Sequence Synthetic polypeptide
HGEGTFTSDLSKQLEEEAARLFIEFLKNGGPSSGAPPPS

SEQ ID NO: 6 - protein, 30 aa, Artificial Sequence
  Description of Artificial Sequence Synthetic polypeptide
HGEGTFTSDLSKQMEEEAVRLFIEWLKNGG

SEQ ID NO: 7 - protein, 30 aa, Artificial Sequence
  Description of Artificial Sequence Synthetic polypeptide
HGEGTFTSDLSKQLEEEAVRLFIEWLKNGG

SEQ ID NO: 8 - protein, 30 aa, Artificial Sequence
  Description of Artificial Sequence Synthetic polypeptide
HGEGTFTSDLSKQLEEEAVRLFIEFLKNGG

SEQ ID NO: 9 - protein, 30 aa, Artificial Sequence
  Description of Artificial Sequence Synthetic polypeptide
HGEGTFTSDLSKQLEEEAARLFIEFLKNGG

SEQ ID NO: 10 - protein, 28 aa, Artificial Sequence
  Description of Artificial Sequence Synthetic peptide
HGEGTFTSDLSKQMEEEAVRLFIEWLKN

SEQ ID NO: 11 - protein, 28 aa, Artificial Sequence
  Description of Artificial Sequence Synthetic peptide
HGEGTFTSDLSKQLEEEAVRLFIEWLKN

SEQ ID NO: 12 - protein, 28 aa, Artificial Sequence
  Description of Artificial Sequence Synthetic peptide
HGEGTFTSDLSKQLEEEAVRLFIEFLKN

SEQ ID NO: 13 - protein, 28 aa, Artificial Sequence
  Description of Artificial Sequence Synthetic peptide
HGEGTFTSDLSKQLEEEAARLFIEFLKN

SEQ ID NO: 14 - protein, 39 aa, Artificial Sequence
  Description of Artificial Sequence Synthetic polypeptide
HGEGTFTSDLSKQLEEKAAKEFIEFLKQGGPSSGAPPPS

SEQ ID NO: 15 - protein, 39 aa, Artificial Sequence
  Description of Artificial Sequence Synthetic polypeptide
HGEGTFTSDLSKQLEEKAAKEFIEWLKQGGPSSGAPPPS

SEQ ID NO: 16 - protein, 39 aa, Artificial Sequence
  Description of Artificial Sequence Synthetic polypeptide
HGEGTFTSDLSKQGEEEAVRLFIEWLKQGGPSSGAPPPS

SEQ ID NO: 17 - protein, 39 aa, Artificial Sequence
  Description of Artificial Sequence Synthetic polypeptide
HGEGTFTSDLSKQLEEEAVRLFIEWLKQGGPSSGAPPPS

SEQ ID NO: 18 - protein, 37 aa, Artificial Sequence
  Description of Artificial Sequence Synthetic polypeptide
HGEFTFTSDLSKQLEEEAVRLFIEWLKQGGPSKEIIS

SEQ ID NO: 19 - protein, 39 aa, Artificial Sequence
  Description of Artificial Sequence Synthetic polypeptide
HGEFTFTSDLSKQLEEKAAKEFIEWLKQGGPSSGAPPPS

SEQ ID NO: 20 - protein, 39 aa, Artificial Sequence
  Description of Artificial Sequence Synthetic polypeptide
HGEGTFTSDLVKILEAEAVRKFIEFLKNGGPSSGAPPPS

SEQ ID NO: 21 - protein, 40 aa, Artificial Sequence
  Description of Artificial Sequence Synthetic polypeptide
HGEGTFTSDLSKQMEEEAVRLFIEWLKNGGPSSGAPPPSK

SEQ ID NO: 22 - protein, 31 aa, Homo sapiens
  C-term may or may not be amidated
HAEGTFTSDVSSYLEGQAAKEFIAWLVKGRG

SEQ ID NO: 23 - protein, 30 aa, Artificial Sequence
  Description of Artificial Sequence Synthetic polypeptide
HAEGTFTSDVSSYLEGQAAKEFIAWLVKGR

SEQ ID NO: 24 - protein, 30 aa, Artificial Sequence
  Description of Artificial Sequence Synthetic polypeptide
HXEGTFTSDVSSYLEGQAAREFIAFLVRXR

SEQ ID NO: 25 - protein, 31 aa, Artificial Sequence
  Description of Artificial Sequence Synthetic polypeptide
HXEGTFTSDVSSYLEXXAAKEFIXWLXXGXX

SEQ ID NO: 26 - protein, 31 aa, Artificial Sequence
  Description of Artificial Sequence Synthetic polypeptide
HAEGTFTSDVSSYLEGQAAKEFIAWLVRGRG

SEQ ID NO: 27 - protein, 31 aa, Artificial Sequence
  Description of Artificial Sequence Synthetic polypeptide
HAEGTFTSDVSSYLEGQAAKEFIEWLVKGRG

SEQ ID NO: 28 - protein, 31 aa, Artificial Sequence
  Description of Artificial Sequence Synthetic polypeptide
HAEGTFTSDVSSYLEKQAAKEFIAWLVKGRG

SEQ ID NO: 29 - protein, 31 aa, Artificial Sequence
  Description of Artificial Sequence Synthetic polypeptide
HGEGTFTSDVSSYLEEQAAKEFIAWLVKGGG

SEQ ID NO: 30 - protein, 31 aa, Artificial Sequence
  Description of Artificial Sequence Synthetic polypeptide
HVEGTFTSDVSSYLEEQAAKEFIAWLVKGGG

SEQ ID NO: 31 - protein, 31 aa, Artificial Sequence
  Description of Artificial Sequence Synthetic polypeptide
HGEGTFTSDVSSYLEEQAAKEFIAWLKNGGG

SEQ ID NO: 32 - protein, 31 aa, Artificial Sequence
  Description of Artificial Sequence Synthetic polypeptide
HVEGTFTSDVSSYLEEQAAKEFIAWLKNGGG

SEQ ID NO: 33 - protein, 31 aa, Artificial Sequence
  Description of Artificial Sequence Synthetic polypeptide
HGEGTFTSDVSSYLEEQAAKEFIAWLVKGGP

SEQ ID NO: 34 - protein, 31 aa, Artificial Sequence
  Description of Artificial Sequence Synthetic polypeptide
HVEGTFTSDVSSYLEEQAAKEFIAWLVKGGP

SEQ ID NO: 35 - protein, 31 aa, Artificial Sequence
  Description of Artificial Sequence Synthetic polypeptide
HGEGTFTSDVSSYLEEQAAKEFIAWLKNGGP

SEQ ID NO: 36 - protein, 31 aa, Artificial Sequence
  Description of Artificial Sequence Synthetic polypeptide
HVEGTFTSDVSSYLEEQAAKEFIAWLKNGGP

SEQ ID NO: 37 - protein, 30 aa, Artificial Sequence
  Description of Artificial Sequence Synthetic polypeptide
HGEGTFTSDVSSYLEEQAAKEFIAWLVKGG

SEQ ID NO: 38 - protein, 30 aa, Artificial Sequence
  Description of Artificial Sequence Synthetic polypeptide
HVEGTFTSDVSSYLEEQAAKEFIAWLVKGG

SEQ ID NO: 39 - protein, 30 aa, Artificial Sequence
  Description of Artificial Sequence Synthetic polypeptide
HVEGTFTSDVSSYLEEQAAKEFIAWLVNGG

SEQ ID NO: 40 - protein, 30 aa, Artificial Sequence
  Description of Artificial Sequence Synthetic polypeptide
HGEGTFTSDVSSYLEEQAAKEFIAWLVNGG

SEQ ID NO: 41 - protein, 230 aa, Artificial Sequence
  Description of Artificial Sequence Synthetic polypeptide
AESKYGPPCPPCPAPXXXGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 30 aa, Artificial Sequence
  Description of Artificial Sequence Synthetic polypeptide
GGGGSGGGGSGGGGSGGGGSGGGGSGGGGS

SEQ ID NO: 43 - protein, 275 aa, Artificial Sequence
  Description of Artificial Sequence Synthetic polypeptide
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 44 - protein, 40 aa, Artificial Sequence
  Description of Artificial Sequence Synthetic polypeptide
HXEGTFTSDVSSYLEXQAAKEFIAWLXKGGPSSGAPPPCC

SEQ ID NO: 45 - protein, 40 aa, Artificial Sequence
  Description of Artificial Sequence Synthetic polypeptide
HVEGTFTSDVSSYLEEQAAKEFIAWLIKGGPSSGAPPPCC

SEQ ID NO: 46 - protein, 39 aa, Artificial Sequence
  Description of Artificial Sequence Synthetic polypeptide
HGEGTFTSDLSKQMEEEAVKLFIEWLKNGGPSSGAPPPS

SEQ ID NO: 47 - protein, 39 aa, Artificial Sequence
  Description of Artificial Sequence Synthetic polypeptide
HGEGTFTSDLSKQMEEEAVRLFIEWLKNGGPKSGAPPPS

SEQ ID NO: 48 - protein, 38 aa, Artificial Sequence
  Description of Artificial Sequence Synthetic polypeptide
GEGTFTSDLSKQMEEEAVKLFIEWLKNGGPSSGAPPPS

SEQ ID NO: 49 - protein, 39 aa, Artificial Sequence
  Description of Artificial Sequence Synthetic polypeptide
HGEGTFTSDLSRQXEEEAVRLFIEWLRNGGPKSGAPPPS

SEQ ID NO: 50 - protein, 39 aa, Artificial Sequence; identical to SEQ ID NO: 46
  Description of Artificial Sequence Synthetic polypeptide

SEQ ID NO: 51 - protein, 39 aa, Artificial Sequence; identical to SEQ ID NO: 46
  Description of Artificial Sequence Synthetic polypeptide

SEQ ID NO: 52 - protein, 39 aa, Artificial Sequence; identical to SEQ ID NO: 46
  Description of Artificial Sequence Synthetic polypeptide

SEQ ID NO: 53 - protein, 39 aa, Artificial Sequence; identical to SEQ ID NO: 46
  Description of Artificial Sequence Synthetic polypeptide

SEQ ID NO: 54 - protein, 40 aa, Artificial Sequence; identical to SEQ ID NO: 21
  Description of Artificial Sequence Synthetic polypeptide

SEQ ID NO: 55 - protein, 40 aa, Artificial Sequence; identical to SEQ ID NO: 21
  Description of Artificial Sequence Synthetic polypeptide

SEQ ID NO: 56 - protein, 40 aa, Artificial Sequence; identical to SEQ ID NO: 21
  Description of Artificial Sequence Synthetic polypeptide

SEQ ID NO: 57 - protein, 40 aa, Artificial Sequence; identical to SEQ ID NO: 21
  Description of Artificial Sequence Synthetic polypeptide

SEQ ID NO: 58 - protein, 40 aa, Artificial Sequence; identical to SEQ ID NO: 21
  Description of Artificial Sequence Synthetic polypeptide

SEQ ID NO: 59 - protein, 40 aa, Artificial Sequence; identical to SEQ ID NO: 21
  Description of Artificial Sequence Synthetic polypeptide

SEQ ID NO: 60 - protein, 30 aa, Artificial Sequence
  Description of Artificial Sequence Synthetic polypeptide
AEGTFTSDVSSYLEGQAAREFIAWLVKGRG

SEQ ID NO: 61 - protein, 30 aa, Artificial Sequence; identical to SEQ ID NO: 60
  Description of Artificial Sequence Synthetic polypeptide

SEQ ID NO: 62 - protein, 32 aa, Artificial Sequence
  Description of Artificial Sequence Synthetic polypeptide
HAEGTFTSDVSSYLEGQAAREFIAWLVRGRGK

SEQ ID NO: 63 - protein, 30 aa, Artificial Sequence
  Description of Artificial Sequence Synthetic polypeptide
HAEGTFTSDVSSYLEGQAAREFIAWLVRGK

SEQ ID NO: 64 - protein, 32 aa, Unknown
  Description of Unknown Exemplary amylin agonist polypeptide
KCNTATCVLGRLSQELHRLQTYPRTNVGSNTY